(19) United States
(12) Patent Application Publication
(10) Pub. No.: US 2011/0076274 A1
(43) Pub. Date: Mar. 31, 2011

(54) MATERIALS AND METHODS RELATING TO A G-PROTEIN COUPLED RECEPTOR

(76) Inventors: Ruth Alexandra Ross, Aberdeen, Scotland (GB); Michael John Rogers, Aberdeen, Scotland (GB); Lesley Ann Ford, Aberdeen, Scotland (GB); Anke Jozefien Roelofs, Aberdeen, Scotland (GB)

(21) Appl. No.: 12/992,312

(22) PCT Filed: May 13, 2009
(86) PCT No.: PCT/GB09/01201

§ 371 Date: Nov. 12, 2010

(30) Foreign Application Priority Data
May 13, 2008 (GB) 0808668.8

(51) Int. Cl. A61K 39/395; G01N 33/574; C12Q 1/68; A61K 39/39; A61K 31/704; A61K 31/675; A61K 31/337; A61P 35/00; A61P 35/04

(52) U.S. Cl. 424/133.1; 435/7.23; 435/6; 424/278.1; 514/34; 514/110; 514/449

(57) ABSTRACT

The present inventors demonstrate that the G-protein coupled receptor 55 (GPR55) is highly expressed in human aggressive breast cancer cells, and that the expression level may be correlated with the invasiveness and metastatic potential of these cells (for example metastasis to bone). In various aspects of the invention there are disclosed diagnostic tools or biomarkers that relate to the metastatic profile of breast cancer tumours. The invention also relates to pharmacological agents targeting this receptor for the purposes of inhibiting progression and spread of breast cancer.

### TECHNICAL FIELD

The present invention relates generally to the endocannabinoid G-protein coupled receptor 55 (GPR55) and uses thereof.

### BACKGROUND ART

The pharmacology of the classic cannabinoid receptors, CB₁ and CB₂, is well established.

However, more recently evidence has emerged for putative novel cannabinoid receptors which have inconsistent pharmacological profiles with the CB₁ and CB₂ receptors.

GPR55 was previously an orphan GPCR. Human GPR55 expression was later found in the ileum, spleen, tonsils, testis and breast and adipose tissue shown in a patent filed by GlaxoSmithKline. CB₁ and CB₂ sequence homology with GPR55 were found to be only 13.5% and 14.4% respectively (Sawzdargo et al., 1999; Brown and Wise, 2003; Ryberg et al., 2007).

One recent study (Johns et al, 2007) sought to test the hypothesis that GPR55 mediates vasodilatation to CBx agonists or atypical cannabinoids. The study showed that GPR55 is activated by atypical cannabinoids but does not mediate their vasodilator effects.

Another recent study (Ryberg et al., 2007) identified the GPR55 coupled G protein as G₁₃ and found downstream activation of the rhoA, rac1 and cdc42 small GTPases. The authors concluded that GPR55 was a candidate for one of the non-CB₁/CB₂ receptors described in the literature.

International patent application WO 01/86305 (Glaxo Group Limited) relates to the identification of modulators of GPR55 activity. A number of assays for modulators are discussed.

International patent application WO 2004/07844 (Astrazeneca UK Limited) relates to the identification of cannabinoid-ligand-type modulators of GPR55 activity. A number of assays for modulators are discussed.

### DISCLOSURE OF THE INVENTION

The present inventors have discovered that GRP55 is expressed in human aggressive breast cancer cells, and that the expression level may be correlated with the invasiveness and metastatic potential of these cells.

Metastasis is the spread of malignant tumors to secondary sites remote from the original or primary tumor. Early stage knowledge of the metastatic potential of a primary tumour can provide clinicians with important information for use in treatment and adjuvant therapy.

Thus, in various aspects of the invention, GPR55 expression may be used as a diagnostic tool or biomarker that relates to the metastatic profile of breast cancer tumours.

The inventors have further discovered that the migration of aggressive breast cells can be modified by pharmacological agents targeting this receptor.

Thus further aspects of the invention relates to the therapeutic targeting of the receptor for the purposes of inhibiting progression and spread of breast cancer.

Also disclosed herein are models of how breast cancer metastases may be directed to bone. Assessment and intervention in this respect (metastasis to bone) forms the basis of yet further aspects of the invention.

Thus in one aspect the invention provides a method of predicting the metastatic potential or determining a metastasis prognosis of breast cancer cells in a biological sample from a subject patient, the method comprising assessing the level of GPR55 expression in the cells.

The method may be preceded by obtaining a sample of the primary tumor (presurgical or intrasurgical biopsy).

As described in more detail levels of “expression” may be detected either from levels of GPR55 nucleic acid or protein. For example protein may be detected in the cell membrane, the endoplasmatic reticulum or the Golgi apparatus (by direct binding or by activity) or nucleic acid may be detected from mRNA encoding GPR55, either directly or indirectly (e.g. via cDNA derived therefrom).

In preferred embodiments the assessment may be an immunoassay based test. For instance, labelled antibodies may be used in an immunoassay to evaluate GPR55 levels in cells or cell membranes. However other methods well known in the art may include the use of fluorescence microscopy, Western blot analysis, m-RNA Northern and slot blot analyses, enzymatic amplification and analysis of m-RNA, fluorescence activated cell sorting, and so on.

In one embodiment the method may comprise the steps of:

(a) contacting a biological sample obtained from the cancer patient with a binding agent that specifically binds to GPR55 or GPR55 mRNA; and
(b) detecting the amount of GPR55 or GPR55 mRNA that binds to the binding agent,
(c) optionally comparing the amount of GPR55 or GPR55 mRNA to a predetermined cut-off value, and thereby determining the metastatic potential of the cancer cells.

In one aspect there is provided a method comprising:

(a) contacting a biological sample obtained from the cancer patient with a binding agent that specifically binds to GPR55; and
(b) detecting the amount of GPR55 that binds to the binding agent,
(c) optionally comparing the amount of GPR55 to a predetermined cut-off value, and thereby determining the metastatic potential of the cancer cells.

The binding agent may specifically bind to an extracellular domain of GPR55 protein. Preferably the agent is an antibody e.g. selected from a monoclonal antibody, a polyclonal antibody, a single chain antibody, a Fab, and an epitope-binding fragment of an antibody. The agent may be detectably labelled e.g. with a radioactive label, a fluorescent label, a chemiluminescent label, and a bioluminescent label.

In another aspect the method may comprise the steps of obtaining a test sample comprising nucleic acid molecules present in a sample of the individual then determining the amount of GPR55 mRNA in the test sample and optionally comparing the amount of GPR55 mRNA in the test sample to a predetermined value.

The step of determining the amount of GPR55 mRNA in the test sample may comprise exposing the test sample to a nucleic acid molecule which hybridizes to a said GPR55 mRNA under stringent conditions. For example the methods may employ a probe of around 30 nucleotides or longer 0.5 M NaHPO₄/7% SDS/1 mM EDTA at 65° C. The stringent conditions may comprise washing in 0.1% SDS/0.1×SSC at 68° C.

More preferably the step of determining the amount of GPR55 mRNA in the test sample entails a specific amplification of the mRNA and then quantitation of the amplified produce e.g. via RT-qPCR analysis as described in the Examples below.

Whichever method for assessing expression is used, the amount determined is preferably normalised compared to a reference gene or protein in cell. The choice of such a gene may be determined by one skilled in the art. In the Examples used below the reference gene is GAPDH. In Ryberg et al. (2007) rb36B4 was used.

The (preferably normalised) expression level of GPR55 may be compared to a control e.g. a human metastatic cell line, and a human non metastatic cell line. A variety of cell lines were tested in the Examples below.

The expression of GPR55 may be relative to CB₁ and CB₂. As noted below, although expression of CB₁ and CB₂ was limited to single cell lines, the results also indicated a correlation between the relative aggressiveness of the cancer cell lines, and the relative level of GPR55 expression.

Methods of the present invention may be used in conjunction with conventional methods of breast cancer diagnosis and staging (e.g. according to the TNM or AJCC systems) and testing for the presence of HER2.

In another aspect the method of predicting the metastatic potential of a breast cancer cells or determining a metastasis prognosis in a biological sample may be used to diagnose the risk of breast cancer, or more specifically the risk or likelihood of metastases in the patient e.g. if GPR55 expression exceeds a predetermined value.

In another aspect the method may be used for selecting an individual for therapy with a compound or for adjuvant therapy e.g. if GPR55 expression exceeds a predetermined value.

In other aspects of the invention, GPR55 and its expression may be used as a biomarker for choosing or monitoring specific therapeutic regimes and chemotherapeutic combinations.

Thus in one aspect the present invention provides a method for monitoring the progression of breast cancer in a patient, comprising the steps of:

(a) assessing the level of GPR55 expression in a biological sample obtained from the patient at a first point;
(b) repeating step (a) using a biological sample obtained from the patient at a subsequent point in time; and
(c) comparing the level of GPR55 expression detected in step (b) to the amount detected in step (a) and thereby monitoring the progression of the cancer in the patient.

For instance the cancer may be determined as progressing if the expression level increases over time, whereas the cancer may not be progressing if the expression level remains constant or decreases with time.

In a preferred embodiment the method is performed as follows:

(a) contacting a biological sample obtained from the patient at a first point in time with a binding agent that specifically binds to GPR55;
(b) detecting in the sample an amount of polypeptide that binds to the binding agent;
(c) repeating steps (a) and (b) using a biological sample obtained from the patient at a subsequent point in time; and
(d) comparing the amount of polypeptide detected in step (c) to the amount detected in step (b) and thereby monitoring the progression of the cancer in the patient, wherein the cancer is progressing if the amount of the polypeptide increases over time, whereas the cancer is not progressing if the amount of the polypeptide remains constant or decreases with time.

The method may be used to monitor the risk of metastases in the patient over time.

The method may also be used for determining whether a therapeutic treatment should be continued, or for monitoring the efficacy of an anticancer therapy of metastases which the patient is undergoing.

The diagnostic, prognostic and other methods described herein may be performed by use of a test kit, and such kits form further aspects of the invention.

Thus the invention provides a test kit for assessing or aiding in any of the diagnostic or prognostic methods described above e.g. for measuring the presence of or amount of GPR55 mRNA in a sample. The kit may comprise:

(a) a nucleic acid molecule comprising at least 30 contiguous nucleotides of the GPR55 nucleotide sequence; and
(b) means for detecting binding of the nucleic acid molecule to the GPR55 mRNA in a sample.

The nucleic acid may be one which directly analyses m-RNA by Northern or other blot analyses, or one (e.g. a primer) which can be used in enzymatic amplification and analysis of m-RNA.

In other aspects a test kit may comprise:

(a) an antibody which selectively binds GPR55;
(b) means for detecting binding of the antibody to GPR55.

In either case the kit may comprise a control sample comprising cells selected from the group consisting of a human metastatic cell line, and a human non-metastatic cell line.

The antibody may be one useful for fluorescence microscopy, Western blot analysis, fluorescence activated cell sorting, or any other immunoassay.

In the light of the disclosure herein it can be seen that inhibition of GPR55 may be useful in the treatment of breast cancer.

Accordingly, a further aspect of the present invention provides a composition comprising a GPR55 inhibitor for use in a method of treatment of breast cancer in a patient. The patient may be one as identified above.

The invention also provides a method of treatment of breast cancer in a patient, which method comprises the step of contacting the tumour with a GPR55 inhibitor.

The invention also provides the use of a GPR55 inhibitor in the preparation of a medicament for the treatment of breast cancer.

A “patient” is a mammal, preferably a human being.

A “GPR55 inhibitor” may achieve its effect by a number of means. For instance, such agents may:

(a) decrease the expression of GPR55, for example via siRNA or other methods described herein using readily available sequence information;
(b) directly antagonise the receptor, or increase receptor desensitisation or receptor breakdown—example antagonists are described herein;
(c) reduce interaction between GPR55 and its endogenous ligands, for example the ligands described in the Examples (preferably by binding GPR55 directly);
(d) reduce GPR55 mediated intracellular signalling, for example by blocking G-protein coupling; and\or
(e) compete with endogenous GPR55 for endogenous ligand binding;

A preferred inhibitor may be a neutralising antibody raised against GPR55. The use of such antibodies represents one feature of the invention.

Other inhibitors include natural or synthetic antagonists e.g. cannabidiol.

Non-naturally inhibitors may be preferred. For example, antagonists may be optimised for highly specific binding and therapeutic use, while maintaining the GPR55 binding affinity. Inhibitors according to the present invention may be provided isolated and\or purified from their natural environment, in substantially pure or homogeneous form, or free or substantially free of other biological material from the species of origin. Where used herein, the term “isolated” encompasses all of these possibilities.

Further, pharmaceutically acceptable active derivatives of such Inhibitors and their use are within the scope of the present invention. Examples of such derivatives include, but are not limited to, salts, solvates, amides, esters, ethers, N-oxides, chemically protected forms, and prodrugs thereof.

Preferably the GPR55 inhibitor is a specific GPR55 inhibitor.

A specific GPR55 inhibitor is one that preferentially targets the GPR55 receptor, for instance with greater inhibitory potential than against the CB1 or CB2 receptors.

The terms “treatment” or “therapy” where used herein refer to any administration of a GPR55 inhibitor intended to alleviate the severity of breast cancer in a subject, and includes treatment intended to cure the disease, provide relief from the symptoms of the disease and to prevent or arrest the development of the disease in an individual at risk from developing the disease or an individual having symptoms indicating the development of the disease in that individual.

A preferred treatment may be such as to minimise metastasis of a primary breast cancer tumour i.e. the methods, uses and compositions of the invention may be applied for the treatment and\or prevention of metastasis of breast cancer.

As noted below, in a cell line with relatively high GPR55 expression putative GPR55 agonists including the endocannabinoid, anandamide (AEA) acted as chemotactic factors. Migration was found to be inhibited by pre-treatment with the GPR55 antagonist, CBD.

Thus the invention provides a method of inhibiting metastasis of a breast cancer tumour, which method comprises the step of contacting the tumour or metastases derived therefrom with a GPR55 inhibitor. Also provided are use of a GPR55 inhibitor to inhibit metastasis of breast cancer and use of a GPR55 inhibitor in the preparation of a medicament for this purpose.

Another preferred treatment may be such as to minimise metastasis of a primary breast cancer tumour to bone i.e. the methods, uses and compositions of the invention may be applied for the treatment and\or prevention of metastatic tumour cell chemotaxis\migration to bone.

Breast cancer metastases are often located in bone tissue. The data herein demonstrate that the endocannabinoids are produced in bone and that they can act as chemotactic factors for metastasis of tumour cells to bone. It is believed that this is via activation of the GPR55 receptor which is up-regulated in aggressive, metastatic breast cancer.

Thus the invention provides a method of inhibiting metastasis of a breast cancer tumour to bone, which method comprises the step of contacting the tumour or metastases derived therefrom with a GPR55 inhibitor. Also provided are use of a GPR55 inhibitor to inhibit metastasis of breast cancer to bone and use of a GPR55 inhibitor in the preparation of a medicament for this purpose.

The present invention provides, in a further aspect, a method of screening for a substance which may be used in any of the therapeutic and inhibitory methods above e.g. inhibition of metastasis of breast cancer and\or inhibition of metastatic tumour cell chemotaxis or migration to bone, the method generally comprising assessing the binding of said substance to the GPR55 receptor or its ability to inhibit expression of the GPR55.

In a preferred aspect the method may comprise:

(i) exposing cells or membranes comprising GPR55 to a test compound for a predetermined length of time;
(ii) detecting the activity or expression of GPR55; and
(iii) comparing the activity or expression of the GPR55 in the cells or membranes treated with the compound relative to activity or expression found in control cells or membranes that were not treated with the compound
wherein compounds with efficacy for use in the methods and treatments of the invention decrease activity or decrease expression of GPR55 relative to the controls.

In one embodiment a method of producing a substance which may be used in any of the therapeutic and inhibitory methods above comprises:

(i) identifying the substance by use of the screening method described above,
(ii) producing said substance.

Other preferred aspects or embodiments of the methods, uses and compositions of the invention will now be discussed:

GPR55 inhibitors, either as disclosed herein or identified using methods as disclosed herein may be present in or formulated into compositions for the pharmaceutical and other uses of the invention described above.

Pharmaceutical compositions according to the present invention may comprise, in addition to the active compound (i.e. the GPR55 inhibitor), a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration.

Thus a further aspect of the present invention provides a process for making a medicament for a treatment described above, the process comprising use of a GPR55 inhibitor e.g. by admixing such an inhibitor with pharmaceutically acceptable components to produce a pharmaceutical composition suitable for administration.

GPR55 inhibitors may be administered alone or in combination with other treatments, either simultaneously or sequentially.

Methods of the invention for inhibiting metastasis of a breast cancer tumour to bone may for example be used in conjunction with treatment with medicines that lower estrogen levels.

For example, by analogy with the use of herceptin, a patient who has tested positive for increased levels of GPR55 expression may be treated by conventional chemotherapy e.g. with doxorubicin and cyclophosphamide preceded by or followed by paclitaxel and a GPR55 inhibitor (and optionally trastuzumab if the patient is HER-2 positive).

Adjuvant therapy for breast cancer following surgery to prevent disease recurrence and progression is specifically embraced by the present invention i.e. the GPR55 inhibitor may be used in a treatment, which treatment comprises surgical intervention e.g. mastectomy. The surgical intervention will precede or follow the use of the GPR55 inhibitor and may not per se form part of the claimed invention.

Thus a combination therapy together with hormonal therapy, chemotherapy, radiotherapy, or thermotherapy is also embraced optionally in conjunction with surgical intervention are included. Other therapies for breast cancer are well known in the art and their use per se does not form part of the present invention. Nevertheless the use of such therapies in combination with GPR55 expression inhibition does form part of the invention.

In accordance with relevant aspects of the present invention a GPR55 inhibitor or composition comprising the same is intended to be administered to individuals. This may be systemically or locally. Administration is preferably in a “therapeutically effective amount”, this being sufficient to show benefit to a subject e.g. reduction, remission, or regression of the breast cancer.

Therefore in a further aspect of the present invention, there the methods of treatment described above may comprise administering to a subject in need of treatment an effective amount of a GPR55 inhibitor.

The actual (effective) amount administered, and rate and time-course of administration, will ultimately be at the discretion of the physician, taking into account the severity of the disease in a particular subject (e.g. a human patient or animal model) and the overall condition of the subject. Suitable dose ranges will typically be in the range of from 0.01 to 20 mg/kg/day, preferably from 0.1 to 10 mg/kg/day w

The use of an antibody raised against GPR55 as an inhibitor according to the invention may involve the administration thereof as a weekly, twice weekly or thrice weekly dose (or more depending upon the severity of the breast cancer) of between 25 mgs and 5000 mgs in injectable form. Alternatively, a slow release device may be used to provide optimal doses to a patient without the need to administer repeated doses.

Definitions and Detailed Methods.

Other aspects of the invention, and definitions used herein, will now be discussed in more detail:

GPR55 Receptor Nucleic Acid

hGPR55 has the EMBL accession no. BC032694. A copy is annexed hereto for ease of reference.

Assessing GPR55 mRNA and Expression

Methods of assessing expression of GPR55 may be conventional in the art, for example as described in Ryberg (2007), or as set out in the Examples below.

GPR55 Receptor Agonists and Antagonists

A wide range of cannabinoids which acted as agonists on the GPR55 receptor are known. These include endogenous cannabinoids such as AEA and components of cannabis such as THC. Synthetic derivatives of naturally occurring cannabinoids such as O-1602 and CP55940 have also been found to act as agonists.

Cannabidiol (CBD), which is also a component of Cannabis sativa, was identified as a GPR55 antagonist.

Table 1 summarises some of these compounds and their effects:

TABLE 1
The actions of selected cannabinoids
on the GPR55, CB₁ and CB₂ receptors.

| | Drug action on receptors | | |
|---|---|---|---|
| Drug | GPR55 | CB₁ | CB₂ |
| O-1602 | Agonist | No action³ | No action |
| | GTPγS | GTPγS | GTPγS |
| | EC₅₀ = 1.4 nM¹ | EC₅₀ > 30,000 nM² | EC₅₀ > 30,000 nM² |
| | GTPγS | | |
| | EC₅₀ = 13 nM² | | |
| JWH015 | Agonist | Poor ligand | Agonist |
| | GTPγS | Kᵢ = 383 nM⁵ | Kᵢ = 14.8 nM⁵ |
| | EC₅₀ = 4 nM⁴ | | |
| LPI | Agonist⁶ | Unknown | Unknown |
| | ERK | | |
| | Phosphorylation | | |
| | EC₅₀ ~ 200 nM⁶ | | |
| AEA | Agonist | Agonist | Agonist |
| | GTPγS | GTPγS | GTPγS |
| | EC₅₀ = 18 nM² | EC₅₀ = 31 nM² | EC₅₀ = 27 nM² |
| CBD | Antagonist² | Antagonist⁷ | Antagonist and |
| | GTPγS | | Inverse |
| | IC₅₀ = 350 nM⁴ | | agonist⁷ |

Compounds such as O-1602 and LPI are therefore thought to be relatively selective GPR55 agonists, whilst AEA has a broad specificity.
References
¹Johns et al., 2007
²Ryberg et al., 2007
³Jarai et al., 1999
⁴Brown, 2007
⁵Showalter et al., 1996
⁶Oka et al., 2007
⁷Thomas et al., 2007

Identification of Novel GPR55 Inhibitors

It is well known that pharmaceutical research leading to the identification of a new drug may involve the screening of very large numbers of candidate substances, both before and even after a lead compound has been found.

More specifically binding assays, antagonist potency, and the activity of “blockers” against G-protein coupling of GPR55 can be assessed and provided (for use in the novel methods and applications described herein) by conventional means, for example as described in Ryberg (2007). Compounds could also be identified using ERK phosphorylation assays (Oka et al, 2008). Example methods are described as follows:

Radioligand Binding Assays

Radioligand binding is initiated by the addition of 5 mg of membrane protein to each well of a 96-well plate containing 50 nM [³H]-(−)-cis-3-[2-hydroxy-4-(1,1-dimethylheptyl)phenyl]-trans-4-(3-hydroxypropyl)cyclohexanol (CP55940) (Tocris, Ellisville, Mo., USA), [3H]-SR141716 (Amersham, Piscataway, N.J., USA) or [³H]-R(p)-[2,3-di-hydro-5-methyl-3-[(morpholinyl)methyl]pyrrolo[1,2,3-de]-1,4-benz-oxazinyl]-(1-naphthalenyl)-methanone-mesylate (WIN55,212-2) (Amersham), sufficient volume of buffer (50 mM Tris-HCl, 5 mM MgCl2, 50 mM NaCl, pH 7.4, 0.1% bovine serum albumin (BSA)) to bring the total volume of each well to 200 μl. Non-specific binding was determined in the presence of 10 μM CP55940 (Tocris), SR141716 and WIN55,212-2 (Tocris). The membranes are incubated at 30° C. for 90 min and the reaction is then terminated by the addition of ice-cold wash buffer (50 mM Tris-HCl, 5 mM MgCl2, 50 mM NaCl, pH 7.4) followed by rapid filtration under vacuum through Printed Filtermat B glass fibre filters (Wallac, Turku, Finland) (0.05% polyethylenimine (PEI)-treated) using a Micro 96 Harvester (Skatron Instruments, Lier, Norway). The filters are dried for 30 min at 50° C., then a paraffin scintillant pad was melted onto the filters and the bound radioactivity was determined using a 1450 Microbeta Trilux (Wallac) scintillation counter.

[35S]-GTPgS Binding Assay

[35S]-Guanosine 50-[g-35S]-triphosphate (GTPgS) binding assays are conducted at 30° C. for 45 min in membrane buffer (100 mM NaCl, 5 mM, 1 mM EDTA, 50 mM HEPES, pH 7.4) containing 0.025 μg μl-1 of membrane protein with 0.01% BSA (fatty-acid free) (Sigma, St Louis, Mo., USA), 10 mM guanosine 50-diphosphate (GDP) (Sigma), 100 mM dithiothreitol (DTT) (Sigma) and 0.53 nM [35S]-GTPgS (Amersham) in a final volume of 200 μl. Non-specific binding was determined in the presence of 20 μM unlabelled GTPgS (Sigma). The reaction was terminated by addition of ice-cold wash buffer (50 mM Tris-HCl, 5 mM MgCl2, 50 mM NaCl, pH 7.4) followed by rapid filtration under vacuum through Wallac GF/B glassfibre filters using a cell harvester (Skatron). The filters were left to dry for 30 min at 50° C., then a paraffin scintillant pad was melted onto the filters and the bound radioactivity was determined using a microbeta scintillation counter (Wallac). Antagonist potency was determined versus an EC80 concentration of CP55940 that was determined empirically on the day of the experiment. Data were fitted using the equation y=A+((B−A)/1+((C/x)̂D))) and the EC50 estimated where A is the non-specific binding, B is the total binding, C is the IC50 and D is the slope.

Peptide and antibody blocking of [35S]-GTPgS binding assays [35S]-GTPgS binding assays were performed as above with additional pre-incubation of membranes with and without peptides or antibodies for the G-protein subunits Gα13, Gαi and Gαs for 15 min at 30° C. (Santa Cruz Biotechnology, Santa Cruz, Calif., USA). Data were analysed using paired t-test (**Po0.05; ***Po0.01).

In this and other aspects, the substances (putative antagonists) may be provided e.g. as the product of a combinatorial library such as are now well known in the art (see e.g. Newton (1997) Expert Opinion Therapeutic Patents, 7(10): 1183-1194).

Essentially, screening methods described herein may be employed analogously to high throughput screens such as those well known in the art, and are based on binding partners—see e.g. WO 200011216 (Bristol-Myers Squibb), which enables fast, throughput screens for evaluation of test compounds that may modulate molecular targets whose specific nucleic acid or amino acid sequences are available, WO 200016231 (Navicyte), which describes a method of screening compound libraries by one or more bioavailability properties such as absorption (such a screen may be used in addition to or as an alternative to a receptor binding based screen); U.S. Pat. No. 6,027,873 (Genencor Intl.), which discloses a method of holding samples for analysis and an apparatus thereof and U.S. Pat. No. 6,007,690 (Aclara Biosciences), which describes integrated microfluidic devices which may be used in high throughput screens and other applications. Other high throughput screens are described in, for example, DE 19835071 (Carl Zeiss; F Hoffman-La Roche), WO 200003805 (CombiChem) and WO 200002899 (Biocept).

Novel compounds for use in the invention (especially GPR55 antagonists) may also be used to design of mimetics. This might be desirable where the active compound is difficult or expensive to synthesise or where it is unsuitable for a particular method of administration. There are several steps commonly taken in the design of a mimetic from a compound having a given target property. Firstly, the particular parts of the compound that are critical and/or important in determining the target property are determined. These parts or residues constituting the active region of the compound are known as its “pharmacophore”. Once the pharmacophore has been found, its structure is modelled according to its physical properties, e.g. stereochemistry, bonding, size and/or charge, using data from a range of sources, e.g. spectroscopic techniques, X-ray diffraction data and NMR. The three dimensional structure may be determined. Computational analysis, similarity mapping (which models the charge and/or volume of a pharmacophore, rather than the bonding between atoms) and other techniques can be used in this modeling process. A template molecule is then selected onto which chemical groups which mimic the pharmacophore can be grafted. The template molecule and the chemical groups grafted on to it can conveniently be selected so that the mimetic is easy to synthesise, is likely to be pharmacologically acceptable, and does not degrade in vivo, while retaining the biological activity of the lead compound.

The screening methods of the invention may utilise cells which are naturally predisposed to express GPR55.

It is also possible to use cells that are not naturally predisposed to express GPR55 provided that such cells are transformed with an expression vector encoding it. Such cells represent preferred test cells for use according to the invention. This is because animal cells or even prokaryotic cells may be transformed to express human GPR55 and therefore represent a good cell model for testing the efficacy of candidate drugs for use in human breast cancer therapy.

International patent application WO 01/86305 (Glaxo Group Limited) relates to the identification of modulators of GPR55 activity. International patent application WO 2004/07844 (Astrazeneca UK Limited) relates to the identification of cannabinoid-ligand-type modulators of GPR55 activity. A number of assays for modulators are discussed in these documents, and the disclosure of these documents inasmuch as it relates to the provision or testing of inhibitors is specifically incorporated herein.

Generally speaking, those skilled in the art are well able to construct vectors and design protocols for recombinant gene expression of GPR55 in cells or cell lines to facilitate screening, and the use of such cells and cell lines in the various identification process embodiments forms one aspect of the present invention. Suitable vectors can be chosen or constructed, containing appropriate regulatory sequences, including promoter sequences, terminator fragments, polyadenylation sequences, enhancer sequences, marker genes and other sequences as appropriate. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, 1992.

The screening methods described herein may also be based upon the use of cell membranes comprising GPR55 or isolated soluble GPR55. Such membranes are preferably derived from the above described cells.

Cells for use in the screening methods of the invention according to the present invention may be contained within an experimental animal (e.g. a mouse or rat) when the method is an in vivo based test. Alternatively the cells may be in a tissue sample (for ex vivo based tests) or the cells may be grown in culture. It will be appreciated that such cells should express, or may be induced to express, functional GPR55.

Compositions of the invention may be formulated for any suitable route and means of administration. Pharmaceutically acceptable carriers or diluents include those used in formulations suitable for oral, rectal, nasal, topical (including buccal and sublingual) or parenteral (including subcutaneous, intramuscular, intravenous, intradermal) administration. The formulations may conveniently be presented in unit dosage form and may be prepared by any of the methods well known in the art of pharmacy. Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

For solid compositions, conventional non-toxic solid carriers include, for example, pharmaceutical grades of mannitol, lactose, cellulose, cellulose derivatives, starch, magnesium stearate, sodium saccharin, talcum, glucose, sucrose, magnesium carbonate, and the like may be used. The active compound (the inhibitor of dexamethasone binding) may be formulated as suppositories using, for example, polyalkylene glycols, acetylated triglycerides and the like, as the carrier. Liquid pharmaceutically administrable compositions can, for example, be prepared by dissolving, dispersing, etc, an active compound as defined above and optional pharmaceutical adjuvants in a carrier, such as, for example, water, saline, aqueous dextrose, glycerol, ethanol, and the like, to thereby form a solution or suspension.

Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound(s) in an amount effective to alleviate the symptoms of the subject being treated.

Dosage forms or compositions containing active ingredient in the range of 0.25 to 95% with the balance made up from non-toxic carrier may be prepared.

For oral administration, a pharmaceutically acceptable non-toxic composition is formed by the incorporation of any of the normally employed excipients, such as, for example, pharmaceutical grades of mannitol, lactose, cellulose, cellulose derivatives, sodium crosscarmellose, starch, magnesium stearate, sodium saccharin, talcum, glucose, sucrose, magnesium carbonate, and the like. Such compositions take the form of solutions, suspensions, tablets, pills, capsules, powders, sustained release formulations and the like. Such compositions may contain 1%-95% active ingredient, more preferably 2-50%, most preferably 5-8%.

Parenteral administration is generally characterized by injection, either subcutaneously, intramuscularly or intravenously. Injectables can be prepared in conventional forms, either as liquid solutions or suspensions, solid forms suitable for solution or suspension in liquid prior to injection, or as emulsions. Suitable excipients are, for example, water, saline, dextrose, glycerol, ethanol or the like.

A more recently devised approach for parenteral administration employs the implantation of a slow-release or sustained-release system, such that a constant level of dosage is maintained. See, e.g., U.S. Pat. No. 3,710,795.

The percentage of active compound contained in such parental compositions is highly dependent on the specific nature thereof, as well as the activity of the compound and the needs of the subject. However, percentages of active ingredient of 0.1% to 10% in solution are employable, and will be higher if the composition is a solid which will be subsequently diluted to the above percentages. Preferably, the composition will comprise 0.2-2% of the active agent in solution.

For intravenous, cutaneous or subcutaneous injection, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

The methods of inhibition described above may in principle be carried out in vitro, for example for research purposes, or other reasons.

If desired, the pharmaceutical composition to be administered may also contain minor amounts of non-toxic auxiliary substances such as wetting or emulsifying agents, pH buffering agents and the like, for example, sodium acetate, sorbitan monolaurate, triethanolamine sodium acetate, sorbitan monolaurate, triethanolamine oleate, etc.

As described herein, antibodies specific for GPR55, and in particular the extracellular domain, have diagnostic and prognostic utility, and may be obtained or provided by methods known in the art.

Antibodies according to the invention may be produced as polyclonal sera by injecting antigen into animals. Preferred polyclonal antibodies may be raised by inoculating an animal (e.g. a rabbit) with antigen using techniques known to the art. The antigen may be the whole GPR55 protein (in glycosylated or non-glycosylated form) or a fragment thereof.

Alternatively the antibody may be monoclonal and raised in mice. Conventional hybridoma techniques may be used to raise the antibodies. The antigen used to generate monoclonal antibodies according to the present invention may be the whole GPR55 protein (in glycosylated or non-glycosylated form) or a fragment thereof.

It is preferred that the antibody is a γ-immunoglobulin (IgG).

It will be appreciated that the variable region of an antibody defines the specificity of the antibody and as such this region should be conserved in functional derivatives of the antibody according to the invention. The regions beyond the variable domains (C-domains) are relatively constant in sequence. It will be appreciated that the characterising feature of antibodies according to the invention is the V_{H} and V_{L} domains. It will be further appreciated that the precise nature of the C_{H} and C_{L} domains is not, on the whole, critical to the invention. In fact preferred antibodies according to the invention may have very different C_{H} and C_{L} domains. Furthermore, as discussed more fully below, preferred antibody functional derivatives may comprise the Variable domains without a C-domain (e.g. scFV antibodies).

An antibody derivative may have 75% sequence identity, more preferably 90% sequence identity and most preferably has at least 95% sequence identity to a monoclonal antibody or specific antibody in a polyclonal mix. It will be appreciated that most sequence variation may occur in the framework regions (FRs) whereas the sequence of the CDRs of the antibodies, and functional derivatives thereof, is most conserved.

A number of preferred embodiments of the seventh aspect of the invention relate to molecules with both Variable and Constant domains. However it will be appreciated that antibody fragments (e.g. scFV antibodies) are also encompassed by the invention that comprise essentially the Variable region of an antibody without any Constant region.

Antibodies generated in one species are known to have several serious drawbacks when used to treat a different species. For instance when murine antibodies are used in humans they tend to have a short circulating half-life in serum and are recognised as foreign proteins by the patient being treated. This leads to the development of an unwanted human anti-mouse (or rat) antibody response. This is particularly troublesome when frequent administrations of the antibody is required as it can enhance the clearance thereof, block its therapeutic effect, and induce hypersensitivity reactions. Accordingly preferred antibodies (if of non-human source) for use in human therapy are humanised.

Monoclonal antibodies are generated by the hybridoma technique which usually involves the generation of non-human mAbs. The technique enables rodent monoclonal antibodies with almost any specificity to be produced. Accordingly preferred embodiments of the invention may use such a technique to develop monoclonal antibodies against GPR55. Although such antibodies are useful therapeutically, it will be appreciated that such antibodies are not ideal therapeutic agents in humans (as suggested above). Ideally, human monoclonal antibodies would be the preferred choice for therapeutic applications. However, the generation of human mAbs using conventional cell fusion techniques has not to date been very successful. The problem of humanisation may be at least partly addressed by engineering antibodies that use V region sequences from non-human (e.g. rodent) mAbs and C region (and ideally FRs from V region) sequences from human antibodies. The resulting ‘engineered’ mAbs are less immunogenic in humans than the rodent mAbs from which they were derived and so are better suited for clinical use.

Humanised antibodies may be chimaeric monoclonal antibodies, in which, using recombinant DNA technology, rodent immunoglobulin constant regions are replaced by the constant regions of human antibodies. The chimaeric H chain and L chain genes may then be cloned into expression vectors containing suitable regulatory elements and induced into mammalian cells in order to produce fully glycosylated antibodies. By choosing an appropriate human H chain C region gene for this process, the biological activity of the antibody may be pre-determined. Such chimaeric antibodies are superior to non-human monoclonal antibodies in that their ability to activate effector functions can be tailored for a specific therapeutic application, and the anti-globulin response they induce is reduced.

Such chimaeric molecules are preferred agents for treating breast cancer according to the present invention. RT-PCR may be used to isolate the V_{H} and V_{L} genes from preferred mAbs, cloned and used to construct a chimaeric version of the mAb possessing human domains.

Further humanisation of antibodies may involve CDR-grafting or reshaping of antibodies. Such antibodies are produced by transplanting the heavy and light chain CDRs of a rodent mAb (which form the antibody's antigen binding site) into the corresponding framework regions of a human antibody.

Proteins and peptide agents according to the present invention (e.g. GPR55 inhibitors) may be subject to degradation by a number of means (such as protease activity at a target site). Such degradation may limit their bioavailability and hence therapeutic utility. There are a number of well-established techniques by which peptide derivatives that have enhanced stability in biological contexts can be designed and produced. Such peptide derivatives may have improved bioavailability as a result of increased resistance to protease-mediated degradation. Preferably, a derivative suitable for use according to the invention is more protease-resistant than the protein or peptide from which it is derived. Protease-resistance of a peptide derivative and the protein or peptide from which it is derived may be evaluated by means of well-known protein degradation assays. The relative values of protease resistance for the peptide derivative and peptide may then be compared.

Peptoid derivatives of proteins and peptides according to the invention may be readily designed from knowledge of the structure of the receptor according to the first aspect of the invention or an agent according to the fourth, fifth or sixth aspect of the invention. Commercially available software may be used to develop peptoid derivatives according to well-established protocols.

Retropeptoids, (in which all amino acids are replaced by peptoid residues in reversed order) are also able to mimic proteins or peptides according to the invention. A retropeptoid is expected to bind in the opposite direction in the ligand-binding groove, as compared to a peptide or peptoid-peptide hybrid containing one peptoid residue. As a result, the side chains of the peptoid residues are able to point in the same direction as the side chains in the original peptide.

A further embodiment of a modified form of peptides or proteins according to the invention comprises D-amino acid forms. In this case, the order of the amino acid residues is reversed. The preparation of peptides using D-amino acids rather than L-amino acids greatly decreases any unwanted breakdown of such derivative by normal metabolic processes, decreasing the amounts of the derivative which needs to be administered, along with the frequency of its administration.

As described above, in different aspects the inhibitor for use in the methods may be a nucleic acid which decreases the expression of GPR55. This may act at the transcriptional or translational level.

In one embodiment this may be an antisense DNA or RNA molecule that will bind to endogenous GPR55 transcripts. Such antisense molecules reduce GPR55 expression and thereby reduce GPR55 mediated activity.

siRNA may also be used as an agent according to the invention. siRNA forms part of a gene silencing mechanism, known as RNA interference (RNAi) which results in the sequence-specific destruction of mRNAs and enables a targeted knockout of gene expression. siRNA used in gene silencing may comprise double stranded RNA of 21 nucleotides length, typically with a 2-nucleotide overhang at each 3′ end. Alternatively, short hairpin RNAs (shRNAs) using sense and antisense sequences connected by a hairpin loop may be used. Both siRNAs and shRNAs can be either chemically synthesized and introduced into cells for transient RNAi or expressed endogenously from a promoter for long-term inhibition of gene expression. siRNA molecules for use as an agent according to the invention may comprise either double stranded RNA of 10-50 nucleotides. Preferably, siRNAs for use as an agent according to the invention comprise 18-30 nucleotides. More preferably, siRNAs for use as an agent according to the invention comprise 21-25 nucleotides. And most preferably, siRNAs for use as an agent according to the invention comprise 21 nucleotides.

siRNA pre-designed for GPR55 is available commercially from Applied Biosystems (850 Lincoln Centre Drive, Foster City, Calif. 94404, USA), currently under the Brand Silencer® Select si RNAs (IDs s17760-s17762).

Accordingly, the vector may comprise a nucleic acid sequence encoding GPR55 suitable for introducing an siRNA into the cell in any of the ways known in the art, for example, as described in any of references cited herein, which references are specifically incorporated herein by reference.

In one embodiment, the vector may comprise a nucleic acid sequence according to the invention in both the sense and antisense orientation, such that when expressed as RNA the sense and antisense sections will associate to form a double stranded RNA. This may for example be a long double stranded RNA (e.g., more than 23 nts) which may be processed in the cell to produce siRNAs (see for example Myers (2003) Nature Biotechnology 21:324-328).

Thus uses of siRNA duplexes containing between 20 and 25 bps, more preferably between 21 and 23 bps, of GPR55 form one aspect of the invention e.g. as produced synthetically, optionally in protected form to prevent degradation. Alternatively siRNA may be produced from a vector, in vitro (for recovery and use) or in vivo.

Any sub-titles herein are included for convenience only, and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1: The relative expression of GPR55 in breast cancer cell lines. Statistical tests could not be performed, and S.E.M could not be calculated for T47D and LNCap as n=1. For all other cell lines, n=3. Error bars are not shown for MDA-MB231 as these values were set as 100%. ns=non-significant and *=P<0.05, compared to MDA-MB231. Statistical analysis performed by Friedman one-way ANOVA, and Dunns post test.

FIG. 2: The expression of CB₁ in breast cancer cell lines relative to NB1. n/d=no detection of CB₁ cDNA. No S.E.M is shown for NB1 as it was set as 100%.

FIG. 3: CB₂ Expression in the breast and prostate cancer cell lines relative to CB₂ cDNA concentrations in undiluted HL60 cDNA. n/d=no detection of CB₂ cDNA.

FIG. 4: Boyden chamber assays used to measure the chemoattractant effect of FBS on serum starved tumour cells relative to mean DMSO vehicle migration. **=P<0.01, tested using paired, non-parametric t-test. Results obtained from one Boyden chamber assay with all wells containing cells from same culture flask and passage number.

FIG. 5: Boyden chamber assays used to measure the effects of FBS and O-1602 on cell migration relative to control. Pooled results from 3 individual Boyden chamber assays, expressed relative to mean cell migration number from all 0.01% DMSO vehicle wells. Individual assays used cells with different passage number. **=P<0.01, comparison with DMSO control, Freidman One-way ANOVA and Dunns post test. FBS vs. O-1602 was non-significant (not shown)

FIG. 6: Boyden chamber assays used to measure the effects of FBS and JWH015 on tumour cell migration compared to vehicle control. Pooled results from 3 individual Boyden chamber assays, expressed relative to mean cell migration number from all 0.01% DMSO vehicle wells. Individual assays used cells with different passage number. ***=P<0.001, **=P<0.01, comparison with DMSO control, Freidman One-way ANOVA with Dunns post test. FBS vs. JWH015, non-significant (not shown)

FIG. 7: Boyden chamber assays used to measure the effects of FBS and Anandamide (AEA) on tumour cell migration compared to vehicle control. Pooled results from 3 individual Boyden chamber assays, expressed relative to mean cell migration number from all 0.01% DMSO vehicle wells. Individual assays used cells with different passage number. ***=P<0.001, **=P<0.01, comparison with DMSO control, Freidman One-way ANOVA with Dunns post test. FBS vs. AEA, non-significant (not shown)

FIG. 8: Boyden chamber assays used to measure the effects of FBS and CBD on tumour cell migration compared to vehicle control. Pooled results from 2 individual Boyden chamber assays, expressed relative to mean cell migration number from all 0.01% DMSO vehicle wells. Individual assays used cells with different passage number. *=P<0.05, ns=non-significant, comparison with DMSO control, Freidman One-way ANOVA with Dunns post test.

FIG. 9: Boyden chamber migration assay. Histograms showing the number of MDA-MB-231 cells which migrated towards FBS following pre-incubation with CBD (1 μM) The data represent mean+/−S.E.M. (n=3-4). *, P<0.05; ***, P<0.001, One-way ANOVA followed by Newman-Keuls multiple comparison tests.

FIG. 10: Cultrex® Cell Invasion Assays. Histogram showing the number of MDA-MB-231 cells which invaded towards FBS following pre-incubation with CBD (1 μM) The data represent mean+/−S.E.M. (n=3). *, P<0.05; **, P<0.01 ***, P<0.001, One-way ANOVA followed by Newman-Keuls multiple comparison tests.

FIG. 11: Boyden chamber migration assay. Histograms showing the number of MDA-MB-231 cells which migrated towards FBS following pre-incubation with LPI (1 μM) The data represent mean+/−S.E.M. (n=3-4). *, P<0.05; ***, P<0.001, One-way ANOVA followed by Newman-Keuls multiple comparison tests.

FIG. 12: Boyden chamber migration assay. Histograms showing the number of MDA-MB-231 cells which migrated towards FBS following pre-incubation with LPI (1 μM) combined with CBD (1 μM). The data represent mean+/−S.E.M. (n=3-4). *, P<0.05; ***, P<0.001, One-way ANOVA followed by Newman-Keuls multiple comparison tests.

FIG. 13: Cultrex® Cell Invasion Assays with MCF7 cells that express low native levels of expression of GPR55 (as shown in FIG. 1). Histograms showing that MCF7 cells transfected with GPR55 invaded towards FBS, whereas empty vector transfected cells did not. CBD significantly attenuated FBS-induced invasion in GPR55 overexpressing cells.

FIG. 14: Boyden chamber migration assay with MCF7 cells that express low native levels of expression of GPR55 (as shown in FIG. 1). MCF7 cells overexpressing GPR55 also migrated towards FBS, whereas empty vector transfected cells did not. In GPR55 overexpressing cells, the FBS-induced migration was significantly enhanced by pre-incubation with 1 μM LPI, and was completely abolished using siRNA to GPR55. The data represent mean+/−S.E.M. (n=2). *, P<0.05; ***, P<0.001, One-way ANOVA followed by Newman-Keuls multiple comparison tests.

FIG. 15: Human M-CSF-dependent macrophages were cultured in the presence of RANKL for 7 days and then treated with 2-AG or anandamide for a further 5 days. Number of F-actin rings in osteoclast cultures, expressed as percentage of vehicle (0.1% DMSO) control±SEM (n=4-7 experiments with five replicates for each). ** P<0.01 compared to control, ANOVA with Dunnett's multiple comparison test.

FIG. 16: 2-AG (A) and anandamide (B) levels following LPS treatment of human osteoclasts. Cells were washed in PBS and treated for 90 minutes with 200 ug/ml LPS. Values represent the mean±SEM from 3 separate donors. Levels of 2-AG in control and LPS-treated cells were not significantly different (Student's paired t-test). Levels of anandamide were undetectable in two control samples which precludes statistical analysis. (C) 2-AG levels in MG-63 cells treated with PTH. Cells were treated for the times indicated with 40 nM or 100 nM PTH. The results are expressed as percentage of control and are the means±SEM (n=2-4 experiments, duplicate samples for each), ** P<0.01 compared to control, ANOVA with Dunnett's multiple comparison test.

### EXAMPLES

The following Methods were used in performance of the Examples herein.

Cell Cultures

MDA-MB231: This is a highly metastatic human breast cancer cell line. In vitro, the MDA-MB-231 cell line has an invasive phenotype.

MCF7: This human breast adenocarcinoma cell line has a less invasive phenotype than MDA-MB231.

T47D: Human breast ductal carcinoma. This cell line is considered non-invasive in vitro due to its inability to penetrate a collagen fibroblast matrix, and its low activity in the Boyden chamber chemoinvasion and chemotaxis assays.

More details of the above cell lines can be found on the University of Texas MD Anderson cancer center “breast cancer cell line database” (The University of Texas M. D. Anderson Cancer Center; 1515 Holcombe Blvd, Houston, Tex. 77030; http://www.mdanderson.org/departments/cancerbiology/)

Cell cultures were maintained in their respective media and, with the exception of MDA-MB231, were incubated with 5% CO₂ at 37° C. MDA-MB231 cells were incubated at 37° C. in a CO₂ free incubator.

Cells were grown in 200 ml culture flasks with 20 ml of their respective medium until confluent. Upon reaching confluence, cell cultures were washed with 5 ml of PBS and dissociated from the flask surface using 5 ml of non-enzymatic cell dissociation solution. 5 ml of their respective medium was added, and fractions of the cell suspension were transferred to new 200 ml cell culture flasks containing 20 ml of medium.

For use in RNA extraction protocols, TRIzol was added to cell lysate and the TRIzol lysate was frozen at −80° C. for later use.

RNA Extraction

The frozen TRIzol lysate were defrosted on ice, and the genomic DNA was sheared by passing the lysate through a 19 G needle several times until they lost their viscosity. 0.2 ml of chloroform was added and the solutions were incubated at room temperature for 5 minutes to allow dissociation of nucleoprotein complexes.

The solutions were centrifuged at 12000 rpm at 4° C. for 15 minutes and the aqueous phases were removed to new eppendorf tubes. After 0.5 ml of isopropanol was added, the solutions were incubated at room temperature for 10 minutes to allow RNA to precipitate. The solutions were then centrifuged at 12000 rpm at 4° C. for 10 minutes and the aqueous phases were removed, leaving visible white RNA pellets.
The RNA pellets were washed by adding 1 ml of 75% ethanol, vortexing and then centrifuging at 12000 rpm at 4° C. for 5 minutes. The aqueous phases were removed, and the samples were allowed to air dry. The RNA samples were diluted in 100 μl of H₂O.

RNA Purification

RNA purification was performed using a Qiagen RNeasy minikit.

350 μl of Buffer RLT was added to each 100 μl RNA sample H₂O solution and mixed. 250 μl of absolute ethanol was added to each RNA sample, and was mixed by pipetting. Each sample was transferred to an RNeasy minicolumn and centrifuged at 12000 rpm at room temperature for 15 seconds. Each column was transferred to a new collection tube. 500 μl of Buffer RPE was added to each RNeasy column and then centrifuged at 12000 rpm at room temperature for 15 seconds. 500 μl of Buffer RPE was added again, and the columns were centrifuged at 12000 rpm at room temperature for 2 minutes. The collection tubes were emptied, and the columns were centrifuged again at 12000 rpm at room temperature for 2 minutes, to ensure complete removal of ethanol.

The RNeasy columns were transferred to 1.5 ml collection tubes, and 30 μl of H₂O was added to each column. The samples were incubated with the H₂O at RT for 2 minutes before being centrifuged at 12000 rpm at RT for 1 minute. The RNeasy columns were transferred to new 1.5 ml collection tubes and a second 30 μl of H₂O was added and incubated for 2 minutes at RT. The columns were then centrifuged at 12000 rpm at RT for 1 minute, but turned 180° on the vertical axis relative to the first H₂O centrifuging step. This was to ensure that RNA unevenly distributed on the RNeasy column surface was not lost.

3 μl aliquots of the RNA samples from the first and second H₂O incubations and centrifuging were made for use in the NanoDrop® Spectrophotometer.

NanoDrop® Spectrophotometry

The NanoDrop® ND-1000 UV-Vis Spectrophotometer and software were used to measure RNA concentration and the 260/230 nm absorbance ratio for each purified RNA sample aliquot.

The spectrophotometer was calibrated with a ‘blank’ of DEPC treated water. Samples were then loaded individually onto the NanoDrop® Spectrophotometer, and absorbance was measured and recorded. An RNA concentration value was calculated by the NanoDrop® software.

The samples that gave the highest RNA concentration for each cell line were used to produce cDNA, and the other samples were discarded.

Purified RNA samples were aliquot into smaller volumes and frozen at −80° C. for later use.

Reverse Transcription

The volume of each RNA sample solution needed to get 2 μg of RNA was calculated using the concentration values established using the NanoDrop® Spectrophotometer. This volume was transferred into 0.5 ml DEPC treated autoclaved tubes, in duplicate for a no reverse transcription control sample to be produced later. These samples were then diluted in DEPC treated water to produce a total volume of 11 μl.

1 μl of random primer (Concentration 2 μg/μl) was added to each RNA sample, and was vortexed and pulsed.

Each sample was then run on a thermocycler for 10 minutes at 70° C. The samples were cooled on ice for 3 minutes and then pulsed to pellet the reaction.

4 μl of 5× reaction buffer, 1 μl of 10 mM dNTP mix and 2 μl of 0.1M DTT were added to each sample. 1 μl of reverse transcriptase was added to one of each cell line RNA sample, leaving another RNA sample for each cell line as a no reverse transcriptase control. The samples were mixed and allowed to incubate for 10 minutes at RT.
The samples were run on a thermocycler at 42° C. for 50 minutes and then 95° C. for 5 minutes. 80 μl of DEPC treated water was added to each reaction sample.

RT-qPCR Analysis

A probe reaction solution was mixed to contain the following quantities of substances in every PCR well used:

1 μl of gene of interest (GPR55, CB₁ or CB₂) TaqMan® probe, 1 μl of VIC labelled GAPDH probe, 10 μl of probe mix and 5 μl of H₂O.

cDNA from GPR55 expressing HEK293 cells, CB₁ expressing NB1 cells, or CB₂ expressing H60 cells were diluted in factors of 1, 10, 100, 1000, 10000 and 10000 to produce a standard curve for these genes of interest.

3 μl of standard curve dilutions were added to wells with the reaction mixture, in duplicate. 3 μl of cDNA from each cell line were added to individual reaction wells in triplicate and 3 μl of no reverse transcription RNA samples for each cell line were added to reaction wells in triplicate to act as a control.

Two blank wells with only the probe reaction mixture and no cDNA solution were also produced as a control. A plastic film was used to cover the wells of the PCR plate, and the plate was centrifuged for one minute to spin down the reaction mixture.

A Roche LightCycler® 480 and software with a Dual Colour Hydrolysis Probe Assay template was used for qPCR analysis, and to determine relative GAPDH and gene of interest (GOI) expression. Double strand cDNA denaturing was done at a temperature of 95° C. and cDNA double strand annealing at 60° C. The denaturing and annealing was done in cycles of 10 and 30 seconds respectively, for a total of 40 cycles. Standard curves for GOI and GAPDH expression were produced from the number of amplification cycles needed to reach a defined threshold of fluorescence in the different factor dilutions of cDNA. This standard curve was then used to calculate arbitrary concentrations of GOI and GAPDH cDNA in the test samples for each cancer cell line.

The average gene of interest and GAPDH concentrations for the triplicate test samples were calculated. The gene of interest concentrations were then normalised by dividing their values by their corresponding GAPDH concentration values.

For GPR55, these normalised concentration values for expression were expressed relative to MDA-MB231 as this cell line was found to have the highest level of GPR55 expression.

Boyden Chamber Cell Migration

Cells used for all chemotaxis assays were bathed in their respective serum-free media and incubated for 19 hours prior to experimentation. The serum starved cells were washed with 5 ml PBS and dissociated from their growth plate using 5 ml of non-enzymatic cell dissociation solution. 5 ml of the respective serum free medium was added after dissociation of the cells, and the cell suspension was transferred to a universal container and centrifuged at 2000 rpm for 5 minutes. The supernatant fluid was discarded and the remaining cell pellet was re-suspended in 2 ml of serum free medium.

10 μl of the resulting cell suspension was transferred to a haemocytometer and the number of cells per ml of suspension was calculated from the counted cell number. Serum free medium was added to the cell suspension in a quantity necessary to bring the suspension to a cell number of 1×10⁶ cells per ml.

Protocol I—Effect of GPR55 Ligand Concentration Gradients on Cell Migration

Solutions of 10% FBS (with 0.01% DMSO vehicle), 0.01% DMSO vehicle control and 1 μM of test drug (O-1602 in 0.01% DMSO vehicle), were prepared. All dilutions were in the appropriate serum-free medium for the cell type being tested. For each assay performed the 24 most central wells of the Boyden chamber were used. The lower chamber wells were loaded with 26 μl of solution to form a slight positive meniscus. A polycarbonate filter with 8 μm pore diameter was placed over the lower wells and the silicon gasket of the Boyden chamber was also put into position. The upper chamber was then fixed in place, and 45 μl of cell suspension was added to each of the 24 upper wells. The Boyden chamber was then incubated at 37° C. (either with or without CO₂ depending on cell type) for 4 hours.

In this protocol, the chemo-attractive effect of each compound concentration gradient was tested with cell migration from the upper well to the lower side of the filter indicating a chemo-attractive effect for the compound.

Protocol II—Effect of Pre-Treatment of Cells with CBD or LPI on Cell Migration

Two aliquots of the 1×10⁶ cells/ml, cell suspension were prepared, and quantities of 0.1% DMSO and 1 μM CBD or LPI (both with 0.1% DMSO vehicle) were added to one aliquot each, to bring the cell suspension concentrations to 0.01% DMSO and 1 μM test compound (with 0.01% DMSO vehicle). The cell suspensions were incubated with each compound for 30 minutes at 37° C. in 5% CO₂.

The assays performed were therefore testing the migration of 0.01% DMSO vehicle treated cells towards 10% FBS (with 0.01% DMSO) and 0.01% DMSO vehicle, and migration of 1 μM or 100 nM CBD treated cells towards 1 μM or 100 nM CBD (with 10% FBS and 0.01% DMSO). The Boyden chamber was incubated at 37° C. with 5% CO₂ for 4 hours.

Fixing and Staining of Migrated Cells

Following incubation, the Boyden chamber was disassembled and the filter was removed and placed in a Petri dish containing 70% ethanol, with the migrated cell side facing up, for 7 minutes. The filter was then transferred to a Petri dish containing distilled water for 2 minutes, with the migrated cell side facing upwards. The non-migrated cell filter side was then drawn over a wiper blade to remove all non-migrated cells, and the filter was allowed to air-dry.

The migrated cell side of the filter was then fixed and stained using a DiffQuik® stain set. The filter was then cut using a surgical scalpel, into 2 pieces with two rows of each column. Each filter piece was mounted onto microscope slides using xylene and p-xylene-bis-pyridinium bromide.

Counting of Migrated Cells

Migrated cells were counted using a Leitz microscope. Individual wells and their borders were visualised using a 10× objective lens and cells were then counted using a 40× objective lens. Cells found in one quarter of three non-overlapping 40× fields within each migration well were counted. The same fields were chosen in every well counted. The total value of cells within each field was calculating by multiplying the number of cells found in one-quarter by 4. The number of cells for each of the 4 fields was then added, and this number was used as a measure for the number of cells migrated within each individual well.

The results for all assays done with each individual drug type were pooled by calculating the mean number of cells migrating in 0.01% DMSO vehicle control wells. The cell number for each individual well was then expressed as a percentage of the vehicle mean.

Cultrex® Cell Invasion Assays

Cell were cultured to 80% confluence then serum starved overnight prior to beginning the assay. On ice, a 0.5×BME stock solution was prepared from the 5× stock in a conical tube and inverted to mix. 100 μl of BME stock solution was aliquoted into each well of the 24 well plate and the chamber incubated at 37° C. overnight. The following morning cells were harvested, washed once using 1× wash buffer, counted, and re-suspended to a density of 5×10⁵ cells ml⁻¹. The top chamber was then carefully aspirated, taking care not to let chambers dry out, and 100 μl of cells per well added to the top chamber. 500 μl of media per well (with or without chemoattractant) was then added to the lower wells of the chamber. The chamber was then assembled and incubated at 37° C. in a humidified environment for 24 hours. The following day the upper chamber was carefully aspirated and washed with 100 μl of 1× wash buffer, and lower wells washed with 500 μl of wash buffer. 12 μl of Calcein-AM solution was added to 12 mL of cell dissociation fluid, and 500 μl of this solution added to the lower chamber. The chamber was then re-assembled and incubated at 37° C. for one hour. The upper chamber was then removed, and dissolved Calcein-AM was read at 485 nm excitation, 520 nm emission.

Tissue Culture and Transient Cell Transfections

MDA-MD-231 cells were maintained in Leibovitz medium supplemented with 10% foetal bovine serum (FBS) and 2 mM L-glutamine, and were incubated at 37° C. in a CO₂ free environment. MCF7 cells were maintained in DMEM supplemented with 10% FBS and 2 mM L-glutamine, and were incubated at 37° C. with 5% CO₂.

MCF7 cells were transfected with GPR55 or empty vector plasmid, GPR55 or negative control siRNA, or a combination of these, by electroporation in an Amaxa Nucleofector II using solution V, and program X-013 or P-020, respectively, according to the recommended protocols for these cell lines. Cells were incubated for 24 h post-transfection, prior to proceeding with the cell chemotaxis assay as described below. For knockdown of GPR55 expression, a set of 4 siRNA duplexes against GPR55 was used (ON-TARGETplus siRNA from Dharmacon, cat. no. LQ-005581-00).

### Example 1

### Receptor Expression Levels in Cell Lines

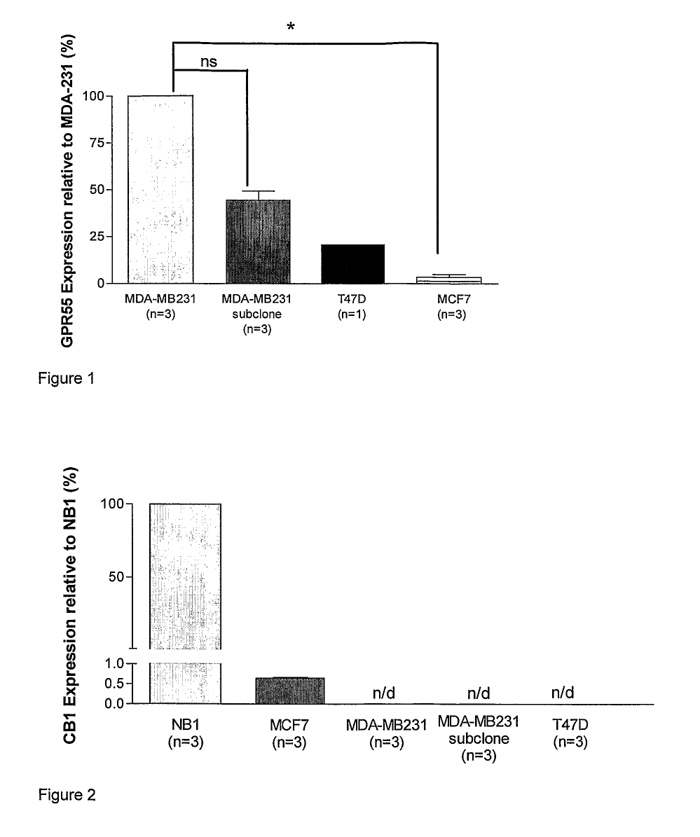

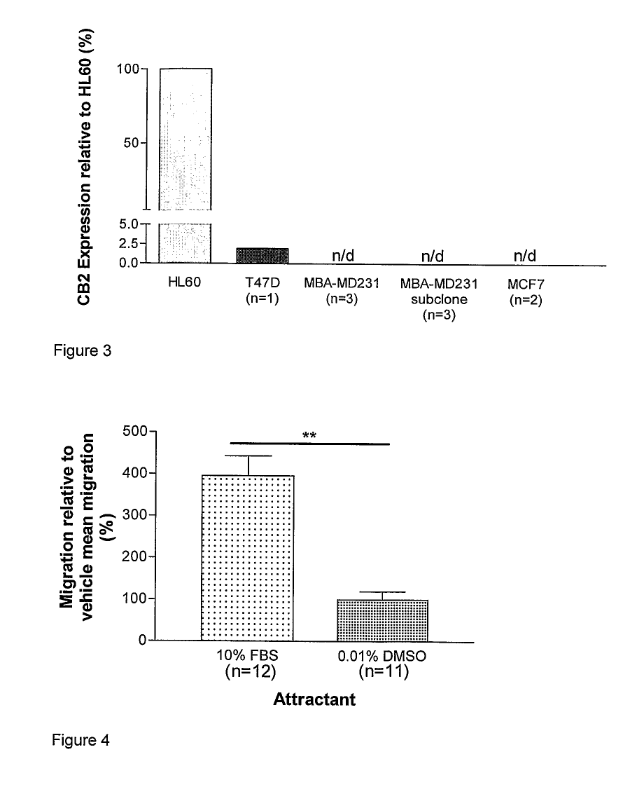

GPR55, CB₁ or CB₂ concentrations in different cell lines were established as described in the methods above using RT-qPCR and then normalised by dividing their value by their respective GAPDH concentration. The MDA-MB231 cell line was found to have the highest level of GPR55 expression and this level was defined as 100%, and the levels of expression in the other cell lines were calculated relative to this. The results for relative GPR55 expression are shown in FIG. 1.

GPR55 expression was found in the breast cancer cell lines. The expression of GPR55 in MCF7 was statistically lower than MDA-MB231.

CB₁ expression was only found in the MCF7 cell line, with no CB₁ cDNA detected in any of the other cancer cell lines. Expression in MCF7 was also found to be relatively low compared to that for undiluted neuroblastoma (NB1) cDNA.

CB₂ expression was only found in the T47D cell line, with no detection in the other cancer cell lines.

Table 2 below shows a summary of the relative expression levels (mean±S.E.M) for the different cannabinoid receptors GPR55, CB₁ and CB₂ found in the different cancer cell lines.

TABLE 2
Summary of cannabinoid receptor expression found
in breast and prostate cancer cell lines.

| | GPR55 | CB₁ | CB₂ |
|---|---|---|---|
| | Expression | Expression | Expression |
| Cancer Cell | Relative to | Relative to | Relative to |
| Line | MDA-MB231 (%) | NB1 (%) | HL60 (%) |
| MDA-MB231 | 100 | n/d | n/d |
| (n = 3) | | | |
| MDA-MB231 | 44.6 ± 4.9 | n/d | n/d |
| subclone (n = 3) | | | |
| MCF7 (n = 3) | 3.5 ± 1.5 | 0.6 ± 0.01 | n/d |
| T47D (n = 1) | 20.7 | n/d | 2.0 |
| LNCap (n = 1) | 4.7 | n/d | n/d |

n/d = no expression detected.

Thus the expression of the classic cannabinoid receptors, CB₁ and CB₂ was found to be largely non-detectable in the breast and prostate cancer cell lines. CB₁ expression was found only in the MCF7 breast cancer cell line which had 0.6% expression relative to the NB1 cell line. It has previously been shown that AEA inhibits cell proliferation in the MCF7 cell line.

For all other cancer cell lines (MDA-MB231, MDA-MB231 subclone, T47D and LNCap), RT-qPCR did not detect any CB₁ expression.

CB₂ expression was found only in the T47D breast cancer cell line, which had 2% expression relative to the HL60 cell line. No CB₂ expression was detected for any of the other cell lines (MDA-MB231, MDA-MB231 subclone, MCF7 and LNCap). This is in line with a study by McKallip et al., in 2005 which also failed to detect CB₂ expression in the MDA-MB231 and MCF7 cell lines.

Expression of the novel cannabinoid receptor GPR55 was found in a wider range of the cancer cells, in contrast to the CB₁ and CB₂ receptors which had a more limited expression throughout the cell lines. MDA-MB231, MDA-MB231 subclone and T47D cells were found to have the highest levels of GPR55 expression, with the MCF7 cells showing a much more limited level of expression. However, in contrast to CB₁ and CB₂, fluorescence due to amplification of GPR55 cDNA reached the defined crossing point for GPR55 in all cancer cell lines. This suggests that GPR55 expression may be relatively higher than CB₁ and CB₂ in the cancer cell lines.

The results indicate a correlation between the aggressiveness of the cancer cell lines and their relative expression of GPR55. MDA-MB231 cells have previously been identified as very invasive in vitro compared, whilst T47D and MCF7 cells are relatively non-invasive (Breast Cancer Cell Line Database, M.D. Anderson Cancer Centre Website; Sommers et al., 1994).

The novel findings described in the present application are consistent with the location of the GPR55 gene, on chromosome 2q, which is a region which has previously been identified as unstable in breast cancer cells (Miller et al., 2003).

Additionally, the ‘signature’ of changed gene expression in at least 70 genes has previously been identified through DNA microarray analysis of primary metastatic cancer cells, with 58 of these genes found to be up-regulated (van't Veer et al., 2002). In addition, in vivo work where MDA-MB231 cells have been injected into mice and the cells metastasizing specifically to bone have been isolated, changes in expression have been found in 46 of these 70 poor-prognosis genes (Minn et al., 2005). Interestingly, one of these signature genes in MDA-MB231 cells, Cdc42 which is activated downstream of GPR55 was shown to be up-regulated (Minn et al., 2005).

Finally, G₁₂ and G₁₃ both belong to the G₁₂ family of heterotrimeric G proteins (Riobo and Manning, 2005) have previously been found to be important transforming factors for cancer cells and for producing aberrant growth (Radhika and Dhanasekaran, 2001). More recently, expression of the α-subunits of these G proteins in breast cancer cells has been shown to increase their invasiveness in vitro. Inhibition of G₁₂ has also been shown to decrease the level of breast cancer cell metastasis in vivo and, interestingly, increased G₁₂ expression has been found in early stage human breast cancer cells taken by biopsy. G₁₂ activation was also found to lead to activation of Rho GTPases in breast cancer cells (Kelly et al., 2006). A recent study (Ryberg et al., 2007) identified the GPR55 coupled G protein as G₁₃.

In summary, the inventors have shown that while expression of CB₁ and CB₂ was limited to single cell lines, GPR55 expression was found in all cancer cell lines tested. The results also indicated a correlation between the relative aggressiveness of the cancer cell lines, and the relative level of GPR55 expression.

### Example 2

### Stimulation of Chemotaxis of MDA-MB231 Subclone in Boyden Chamber Assays

FBS vs. Vehicle

Using serum starved cells, the chemoattractant effect of FBS (with 0.01% DMSO) was tested against its 0.01% DMSO vehicle control, which would later also be used as a vehicle for the test cannabinoid compounds. The results of this assay are shown in FIG. 4.

FIG. 4 shows that tumour cell migration was significantly increased in wells containing 10% FBS (with 0.01% DMSO), with the mean migration almost 4-fold of that observed in the presence of the 0.01% DMSO vehicle control alone.

O-1602

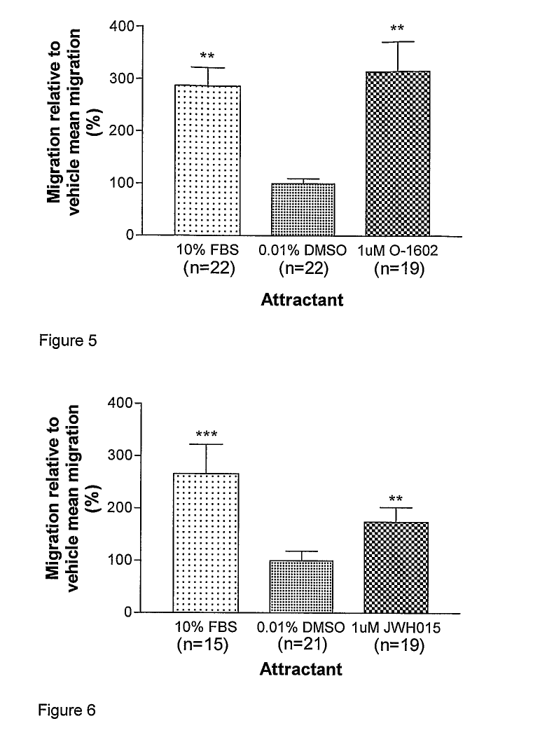

FIG. 5 shows the results of Boyden chamber chemotaxis assays performed with the putative GPR55 agonist, O-1602 (made up in 0.01% DMSO). FBS (with 0.01% DMSO) and 0.01% DMSO vehicle were used as positive and negative controls respectively.

1 μM of the putative GPR55 agonist O-1602 was found to have chemoattractant properties for the cell line, with a significant increase in cell migration compared to vehicle control.

JWH015

FIG. 6 shows the chemoattractant effects of the GPR55 agonist, JWH015 (with 0.01% DMSO) on serum-starved cells, using FBS (with 0.01% DMSO) and 0.01% DMSO as positive and negative controls respectively.

The proposed GPR55 agonist JWH015 was found to have a positive effect on cell migration, with a significant increase in cell number compared to control.

AEA

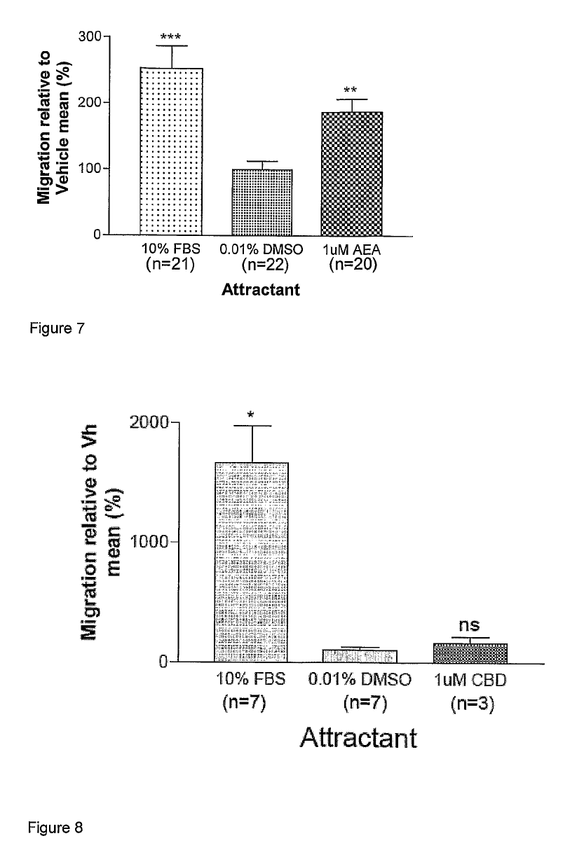

FIG. 7 shows the effect of the endogenous cannabinoid AEA (with 0.01% DMSO) on serum starved cell migration. FBS (with 0.01% DMSO) and 0.01% DMSO vehicle were used as positive and negative controls respectively.

The endogenous cannabinoid ligand AEA was also found to have a positive effect on cell migration with a significant increase in cell number, compared to control. FBS results were consistent with those found in all previous migration assays.

### Example 3

### Inhibition of Chemotaxis of MDA-MB231 in Boyden Chamber Chemotaxis and Invasion Assays by CBD

FIG. 8 shows the effects of CBD (with 0.01% DMSO) on the migration of serum starved cells. FBS (with 0.01% DMSO) and 0.01% DMSO vehicle were used as positive and negative controls respectively. CBD was found to have no effect on tumour cell migration, with no significant difference in number of migrated cells compared to control.

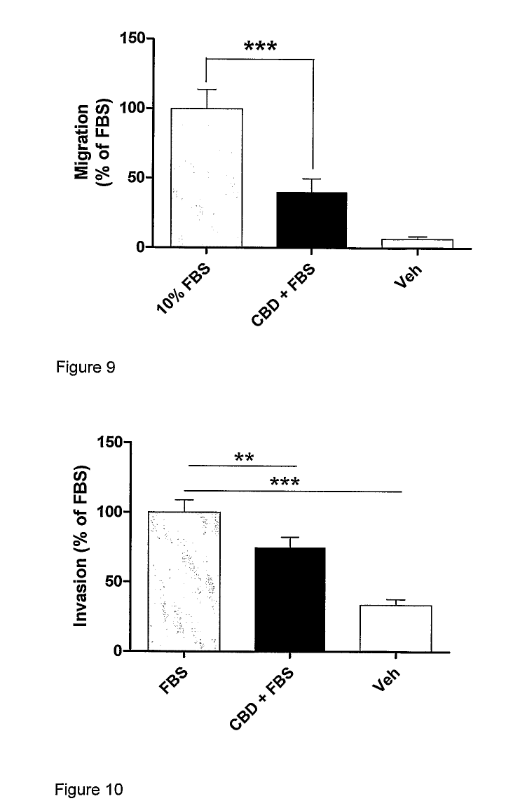

MDA-MB231 cells were pre-incubated with the GPR55 antagonist, CBD (1 μM with 0.01% DMSO) or 0.01% DMSO vehicle, and the effects on migration towards an FBS concentration gradient were measured using 3 individual Boyden chamber migration assays. 1 μM CBD (with 0.01% DMSO) was also placed in the migration wells of the CBD treated cells along with 10% FBS (with 0.01% DMSO), so that no CBD concentration gradient existed. The effects on FBS induced migration would therefore be due to the CBD pre-incubation. FIG. 9 shows the results indicating that CBD significantly inhibits tumour cell chemotaxis to FBS.

CBD (1 μM) pre-incubation was also found to have an inhibitory effect on tumour cell invasion towards FBS (FIG. 10). The pro-invasion effect of FBS was significantly attenuated with CBD pre-treatment.

### Example 4

### Enhancement of Chemotaxis of MDA-MB231 in Boyden Chamber Chemotaxis Assays by LPI

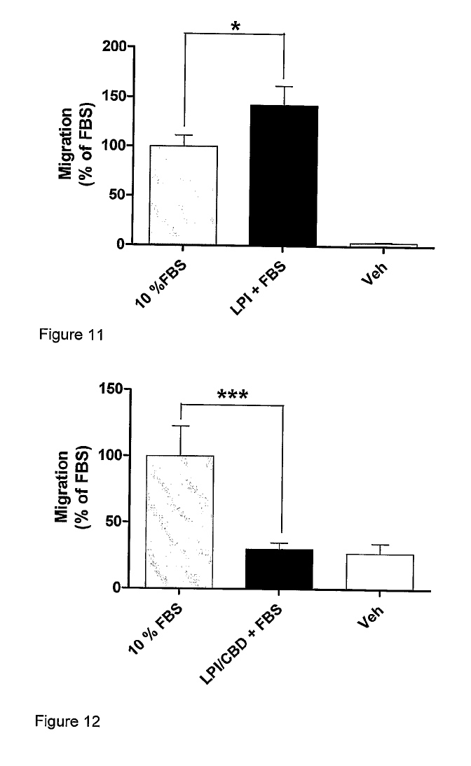

MDA-MB231 cells were pre-incubated with the GPR55 agonist, LPI (1 μM with 0.01% DMSO) or 0.01% DMSO vehicle, and the effects on migration towards an FBS concentration gradient were measured using 3 individual Boyden chamber migration assays. 1 μM LPI (with 0.01% DMSO) was also placed in the migration wells of the LPI treated cells along with 10% FBS (with 0.01% DMSO), so that no LPI concentration gradient existed. The effects on FBS induced migration would therefore be due to the LPI pre-incubation. FIG. 11 shows the results indicating that LPI significantly increases tumour cell chemotaxis to FBS. The effect of LPI is prevented by co-incubation with the GPR55 antagonist, CBD (FIG. 12).

### Example 5

### Over Expression of GPR55 Induces an Invasive Phenotype in MCF7 Cells and the Effects of CBD and LPI on Tumour Cell Invasion and Migration are Mediated by GPR55

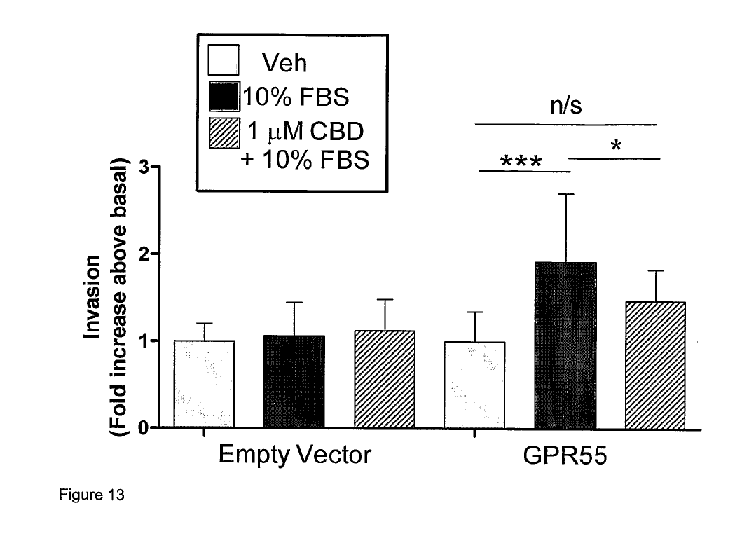

MCF7 cells exhibit very low levels of native expression of GPR55 (FIG. 1). MCF7 cells, transfected with empty vector did not migrate towards FBS in a Cultrex® Cell Invasion Assay (FIG. 13). In contrast, in MCF7 cells transiently over-expressing GPR55 there was a significant invasion of the cells towards FBS (FIG. 13). The GPR55 antagonist, CBD had no effect on basal invasion of control (empty vector transfected) MCF7 cells; however, CBD significantly inhibited FBS-induced invasion in GPR55 over-expressing MCF7 cells (FIG. 13). These results indicate that the inhibition of tumour cell invasion by CBD is mediated by GPR55.

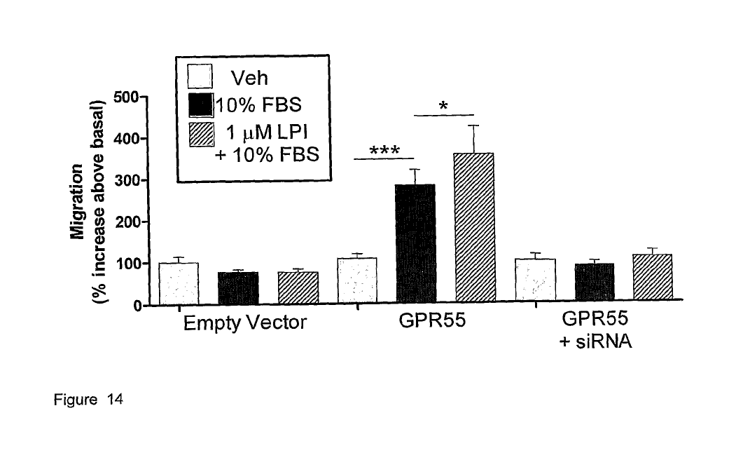

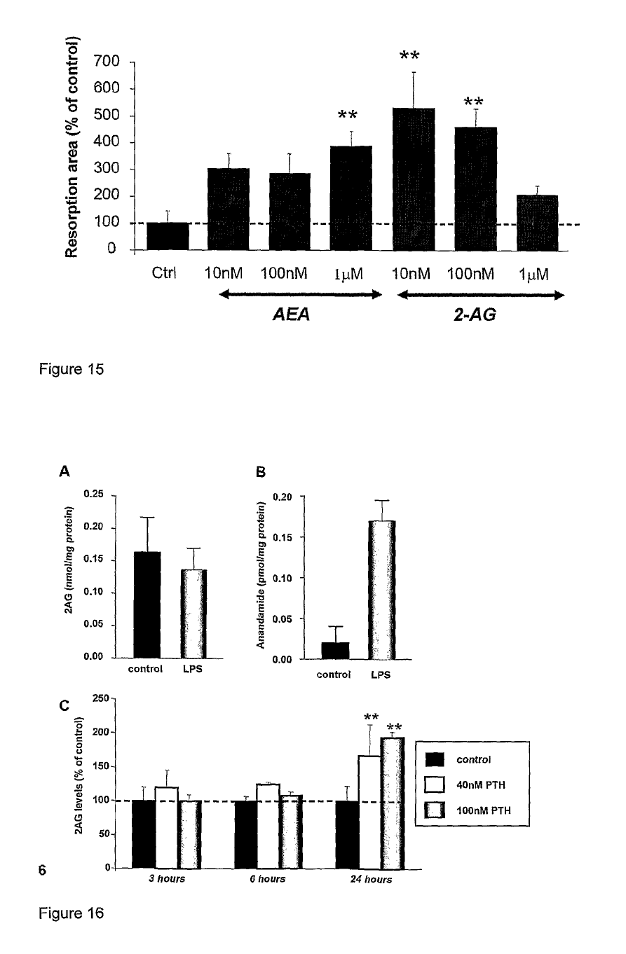

Similarly, in a Boyden chamber chemotaxis assay, vector transfected MCF7 cells did not migrate towards FBS and were unaffected by LPI (FIG. 14). However, in MCF7 cells transiently over-expressing GPR55 (FIG. 14) LPI enhanced FBS-induced migration in a similar manner to that observed in MDA-MB-231 cells. Furthermore, the migration of GPR55 expressing MCF7 cells towards LPI was completely prevented by siRNA to GPR55, confirming that the stimulation of cell migration by LPI is mediated by GPR55.

Summary

1. The breast tumour cell line MDA-MB-231 expresses GPR55; MCF7 cells express significantly lower levels of this receptor.

2. The putative GPR55 agonists O-1602, AEA and JWH015 were all found to promote migration of the GPR55 expressing tumour cells.

3. Pre-treatment of the MDA-MB-231 cells with the GPR55 antagonist, CBD (1 μM) for 30 minutes was found to abolish the migration and attenuate the invasion of the cells towards FBS. In contrast, pre-treatment of the MDA-MB-231 cells with the GPR55 agonist, LPI (1 μM) for 30 minutes was found to increase the migration of the cells towards FBS. Note that in an alamarBlue® assay to assess the viability of the cells CBD had no cytotoxic action on these cells with concentrations up to 10 μM treatment for 4 hours (migration assay) or 1 μM treatment for 24 hours (invasion assay).

4. MCF7 cells express low levels of native expression of GPR55 and are non-invasive. Over expression of GPR55 induces an invasive phenotype in MCF7 cells and the effects of CBD and LPI on tumour cell invasion and migration are mediated by GPR55.

### Example 6

### Endocannabinoid Production in Bone Cells

The Endocannabinoids 2-AG and Anandamide Stimulate Cell Polarisation and Resorption by Human Osteoclasts

Treatment of human osteoclasts with 10 nM and 100 nM 2-AG resulted in a significant increase in cell polarisation i.e. number of F-actin rings (FIG. 11) and resorptive activity, although this stimulatory effect was lost at a higher concentration (1 μM). Treatment of osteoclast cultures with 10 nM to 1 μM anandamide did not result in a significant change in F-actin ring number (data not shown) but did significantly stimulate resorption (FIG. 11).

Endocannabinoids are Produced by Human Osteoblasts and Osteoclasts

Basal levels of 2-AG and anandamide were measured in human osteoblast-like cell lines, macrophages and osteoclasts. 2-AG was detected in MG-63 (0.11±0.02 nmol/mg protein) and TE85 (0.28±0.03 nmol/mg protein) osteoblast-like cells, and in human macrophages (0.43±0.14 nmol/mg protein) and osteoclasts (0.11±0.02 nmol/mg protein), with significantly lower levels in osteoclasts than in macrophages. Anandamide was detected in MG-63 cells (0.12±0.01 pmol/mg protein) and human osteoclasts (0.13±0.02 pmol/mg protein, 4 out of 6 donors), but not in TE85 osteoblast-like cells or human macrophages.

Treatment with LPS did not alter the levels of 2-AG detected in human osteoclasts from 3 separate donors (0.06±0.01 to 0.0.9±0.01 nmol/mg protein, 0.24±0.08 to 0.20±0.02 nmol/mg protein, 0.19±0.04 to 0.12±0.04 nmol/mg protein, FIG. 12A) but did cause an increase in the amount of anandamide detected (0.06±0.01 to 0.14±0.03 pmol/mg protein, undetectable to 0.15±0.09 pmol/mg protein or 0.22±0.07 pmol/mg protein, FIG. 12B). Prolonged treatment of MG-63 cells with PTH resulted in a significant increase in the levels of 2-AG detected (FIG. 12C) and no change in the levels of anandamide detected (data not shown).

Breast cancer metastases are often located in bone tissue. The data demonstrate that the endocannabinoids are produced in bone and that these are produced in response to activation of the PTH receptor, which is involved in tumour cell migration. Furthermore, the endocannabinoids act as chemotactic factors for tumour cells, thus may act as factors involved in the metastasis of tumour cells to bone via activation of the GPR55 receptor which is upregulated in aggressive, metastatic breast cancer.

### REFERENCES

Breast Cancer Cell Line Database, M.D. Anderson Cancer Centre Website, University of Texas. http://www.mdanderson.org/departments/cancerbiology/dIndex.cfm?pn=31062032-BOEB-11D4-80FB00508B603A14 (Accessed 7 Apr. 2008)

Brown A., Wise A., Inventors. GlaxoSmithKline, assignee (2003). U.S. Pat. No. 0,113,814

Brown A. J., (2007). Novel cannabinoid receptors. British Journal of Pharmacology, 152, 567-575

Jarai Z., Wagner J. A., Varga K., et al., (1999). Cannabinoid-induced mesenteric vasodilation through en endothelial site distinct from CB1 or CB2 receptors. Proceedings of the National Academy of Sciences USA, 96 (24), 14136-14141

Johns D. G., Behm D. J., Walker D. J., Ao Z., Shapland E. M., Daniels D. A., Riddick M., Dowell S., Staton P. C., Green P., Bao W., Aiyar N., Yue T-L., Brown A. J., Morrison A. D., Douglas S. A., (2007). The novel endocannabinoid receptor GPR55 is activated by atypical cannabinoids but does not mediate their vasodilator effects. British Journal of Pharmacology, 152, 825-831

Kelly P., Moeller B. J., Juneja J., Booden M. A., Der C. J., Daaka Y., Dewhirst M. W., Fields T. A., Casey P. J., (2006). The G12 family of heterotrimeric G proteins promotes breast cancer invasion and metastasis. Proceedings of the National Academy of Sciences of the USA, 103 (21), 8173-8178

McKallip R. J., Nagarkatti M., Nagarkatti P. S., (2005). Δ-9-Tetrahydrocannabinol enhances breast cancer growth and metastasis by suppression of the antitumor immune response. The Journal of Immunology, 174 (6), 3281-3289

Miller B. J., Wang D., Krahe R., Wright F. A., (2003). Pooled analysis of loss of heterozygosity in breast cancer: a genome scan provides comparative evidence for multiple tumor suppressors and identifies novel candidate regions. American Journal of Human Genetics, 73 (4), 748-767

Minn A. J., Kang Y., Serganova I., Gupta G. P., Giri D. D., Doubrovin M., Ponomarev V., Gerald W. L., Blasberg R., Massague J., (2005). Distinct organ-specific metastatic potential of individual breast cancer cells and primary tumours. Journal of Clinical Investigation, 115 (1), 44-55

Oka S., Nakajima K., Yamashita A., Kishimoto S., Sugiura T., (2007). Identification of GPR55 as a lysophosphatidylinositol receptor. Biochemical and Biophysical Research Communications, 362 (4), 928-934

Ryberg E., Larsson N., Sjögren S., Hjorth S., Hermansson N-O., Leonova J., Elebring T., Nilsson K., Drmota T., Greasley P. J., (2007). The orphan receptor GPR55 is a novel cannabinoid receptor. British Journal of Pharmacology, 152, 1092-1101

Sawzdargo M., Nguyen T., Lee D. K., Lynch K. R., Cheng R., Heng H. H. Q., George S. R., O'Dowd B. F., (1999). Identification and cloning of three novel human G protein-coupled receptor genes GPR52, ψGPR53 and GPR55: GPR55 is extensively expressed in human brain. Molecular Brain Research, 64, 193-198

Sommers C. L., Byers S. W., Thompson E. W., Torri J. A., Gelmann E. P., (1994). Differentiation state and invasiveness of human breast cancer cell lines. Breast Cancer Research and Treatment, 31, 325-335

Sawzdargo M., Nguyen T., Lee D. K., Lynch K. R., Cheng R., Heng H. H. Q., George S. R., O'Dowd B. F., (1999). Identification and cloning of three novel human G protein-coupled receptor genes GPR52, ψGPR53 and GPR55: GPR55 is extensively expressed in human brain. Molecular Brain Research, 64, 193-198

Showalter V. M., Compton D. R., Martin B. R., Abood M. E., (1996). Evaluation of binding in a transfected cell line expressing a peripheral cannabinoid receptor (CB2): Identification of cannabinoid receptor subtype selective ligands. The Journal of Pharmacology and Experimental Therapeutics, 278 (3), 989-999

Thomas A., Baillie G. L., Phillips A. M., Razdan R. K., Ross R. A., Pertwee R. G., (2007). Cannabidiol displays unexpectedly high potency as an antagonist of CB₁ and CB₂ receptor agonists in vitro. British Journal of Pharmacology, 150, 613-623

van't Veer L. J., Dai H., van de Vijver M. J., et al., (2002). Gene expression profiling predicts the clinical outcome of breast cancer. Nature, 415, 530-536

| SEQUENCE ANNEX - Homo sapiens G protein-coupled receptor 55, mRNA | |
|---|---|
| ID BC032694; SV 1; linear; mRNA; STD; HUM; 2629 BP. | |
| XX | |
| AC BC032694; | |
| XX | |
| DT 27-JUN-2002 (Rel. 72, Created) | |
| DT 29-SEP-2006 (Rel. 89, Last updated, Version 15) | |
| XX | |
| DE Homo sapiens G protein-coupled receptor 55, mRNA (cDNA clone MGC:45233 | |
| DE IMAGE:5228678), complete cds. | |
| XX | |
| KW MGC. | |
| XX | |
| OS Homo sapiens (human) | |
| OC Eukaryota; Metazoa; Chordata; Craniata; Vertebrata; Euteleostomi; Mammalia; | |
| OC Eutheria; Euarchontoglires; Primates; Haplorrhini; Catarrhini; Hominidae; | |
| OC Homo. | |
| XX | |
| RN [1] | |
| RP 1-2629 | |
| RX DOI; 10.1073/pnas.242603899 | |
| RX PUBMED; 12477932. | |
| RG Mammalian Gene Collection Program Team | |
| RA Strausberg R.L., Feingold E.A., Grouse L.H., Derge J.G., Klausner R.D., | |
| RA Collins F.S., Wagner L., Shenmen C.M., Schuler G.D., Altschul S.F., | |
| RA Zeeberg B., Buetow K.H., Schaefer C.F., Bhat N.K., Hopkins R.F., Jordan H., | |
| RA Moore T., Max S.I., Wang J., Hsieh F., Diatchenko L., Marusina K., | |
| RA Farmer A.A., Rubin G.M., Hong L., Stapleton M., Soares M.D., Bonaldo M.F., | |
| RA Casavant T.L., Scheetz T.E., Brownstein M.J., Usdin T.B., Toshiyuki S., | |
| RA Carninci P., Prange C., Raha S.S., Loquellano N.A., Peters G.J., | |
| RA Abramson R.D., Mullahy S.J., Bosak S.A., McEwan P.J., McKernan K.J., | |
| RA Malek J.A., Gunaratne P.H., Richards S., Worley K.C., Hale S., Garcia A.M., | |
| RA Gay L.J., Hulyk S.W., Villalon D.K., Muzny D.M., Sodergren E.J., Lu X., | |
| RA Gibbs R.A., Fahey J., Helton E., Ketteman M., Madan A., Rodrigues S., | |
| RA Sanchez A., Whiting M., Madan A., Young A.C., Shevchenko Y., Bouffard G.G., | |
| RA Blakesley R.W., Touchman J.W., Green E.D., Dickson M.C., Rodriguez A.C., | |
| RA Grimwood J., Schmutz J., Myers R.M., Butterfield Y.S., Krzywinski M.I., | |
| RA Skalska U., Smailus D.E., Schnerch A., Schein J.E., Jones S.J., Marra M.A.; | |
| RT “Generation and initial analysis of more than 15,000 full-length human and | |
| RT mouse cDNA sequences”; | |
| RL Proc. Natl. Acad. Sci. U.S.A. 99(26):16899-16903(2002). | |
| XX | |
| RN [2] | |
| RC NIH-MGC Project URL: http://mgc.nci.nih.gov | |
| RP 1-2629 | |
| RG NIH MGC Project | |
| RA ; | |
| RT ; | |
| RL Submitted (06-JUN-2002) to the EMBL/GenBank/DDBJ databases. | |
| RL National Institutes of Health, Mammalian Gene Collection (MGC), Bethesda, | |
| RL MD 20892-2590, USA | |
| XX | |
| DR ASTD; TRAN00000117697. | |
| DR H-InvDB; HIT0000416741. | |
| DR RZPD; IMAGp998K1511575. | |
| DR RZPD; IRAKp961B2269Q. | |
| XX | |
| CC Contact: MGC help desk | |
| CC Email: cgapbs-r@mail.nih.gov | |
| CC Tissue Procurement: Life Technologies, Inc. | |
| CC cDNA Library Preparation: Life Technologies, Inc. | |
| CC cDNA Library Arrayed by: The I.M.A.G.E. Consortium (LLNL) | |
| CC DNA Sequencing by: National Institutes of Health Intramural | |
| CC Sequencing Center (NISC), | |
| CC Gaithersburg, Maryland; | |
| CC Web site: http://www.nisc.nih.gov/ | |
| CC Contact: nisc_mgc@nhgri.nih.gov | |
| CC Akhter,N., Ayele,K., Beckstrom-Sternberg,S.M., Benjamin,B., | |
| CC Blakesley,R.W., Bouffard,G.G., Breen,K., Brinkley,C., Brooks,S., | |
| CC Dietrich,N.L., Granite,S., Guan,X., Gupta,J., Haghighi,P., | |
| CC Hansen,N., Ho,S.-L., Karlins,E., Kwong,P., Laric,P., Legaspi,R., | |
| CC Maduro,Q.L., Masiello,C., Maskeri,B., Mastrian,S.D.,McCloskey,J.C., | |
| CC McDowell,J., Pearson,R., Stantripop,S., Thomas,P.J., Touchman,J.W., | |
| CC Tsurgeon,C., Vogt,J.L., Walker,M.A., Wetherby,K.D., Wiggins,L., | |
| CC Young,A., Zhang,L.-H. and Green,E.D. | |
| CC Clone distribution: MGC clone distribution information can be found | |
| CC through the I.M.A.G.E. Consortium/LLNL at: http://image.llnl.gov | |
| CC Series: IRAK Plate: 69 Row: b Column: 22 | |
| CC This clone was selected for full length sequencing because it | |
| CC passed the following selection criteria: matched mRNA gi: 33695106. | |
| CC Differences found between this sequence and the human reference | |
| CC genome (build 36) are described in misc_difference features below. | |
| XX | |
| FH Key Location/Qualifiers | |
| FH | |
| FT source 1 . . . 2629 | |
| FT /organism=“Homo sapiens” | |
| FT /lab_host=“DH10B” | |
| FT /mol_type=“mRNA” | |
| FT /clone_lib=“NIH_MGC_120” | |
| FT /clone=“MGC:45233 IMAGE:5228678” | |
| FT /tissue_type=“Pancreas, Spleen, adult pooled” | |
| FT /note=“Vector: pCMV-SPORT6” | |
| FT /db_xref=“taxon:9606” | |
| FT /db_xref=“RZPD:IMAGp998K1511575” | |
| FT /db_xref=“RZPD:IRAKp961B2269Q” | |
| FT gene 1 . . . 2629 | |
| FT /gene=“GPR55” | |
| FT CDS 194 . . . 1153 | |
| FT /codon_start=1 | |
| FT /gene=“GPR55” | |
| FT /product=“GPR55 protein” | |
| FT /db_xref=“GDB:9955631” | |
| FT /db_xref=“GOA:Q9Y2T6” | |
| FT /db_xref=“HGNC:4511” | |
| FT /db_xref=“InterPro:IPR000276” | |
| FT /db_xref=“UniProtKB/Swiss-Prot:Q9Y2T6” | |
| FT /protein_id=“AAH32694.1” | |
| FT translation=“MSQQNTSGDCLFDGVNELMKTLQFAVHIPTFVLGLLLNLLAIHGF | |
| FT TFLKNRWPDYAATSIYMINLAVFDLLLVLSLPFKMVLSQVQSPFPSLCTLVECLYFVS | |
| FT MYGSVFTICFISMDRFLAIRYPLLVSHLRSPRKIFGICCTIWVLVWTGSIPIYSFHGK | |
| FT VKYMCFHNMSDDTWSAKVFFPLEVFGFLLPMGIMGFCCSRSIHILLGRRDHTQDWVQQ | |
| FT KACIYSIAASLAVFVVSFLPVHLGFFLQFLVRNSFIVECRAKQSISFFLQLSMCFSNV | |
| FT NCCLDVFCYYFVIKEFRMNIRAHRPSRVQLVLQDTTISRG” | |
| FT misc_difference 1788 | |
| FT /gene=“GPR55” | |
| FT /note=“1 base in cDNA is not found in the human genome.” | |
| FT misc_difference 2529 . . . 2629 | |
| FT /gene=“GPR55” | |
| FT /note=“polyA tail: 101 bases do not align to the human | |
| FT genome.” | |
| XX | |
| SQ Sequence 2629 BP; 675 A; 688 C; 686 G; 580 T; 0 other; | |
| caggcctgga ggagaaatga catctctcag ccctctcagc tgcaccggac caccaacagt | 60 |
| tgtgattcaa tgggcatgaa ttgctgtgtg atgctgggga ggtgtttgtg attcttgaca | 120 |
| aagtcatttg aatccatcac ttcaagagag tgaaaggagc cccgtctgat ctgttggtgt | 180 |
| tgtaggaaga aacatgagtc agcaaaacac cagtggggac tgcctgtttg acggtgtcaa | 240 |
| cgagctgatg aaaaccctac agtttgcagt ccacatcccc accttcgtcc tgggcctgct | 300 |
| cctcaacctg ctggccatcc atggcttcag caccttcctt aagaacaggt ggcccgatta | 360 |
| tgctgccacc tccatctaca tgatcaacct ggcagtcttt gacctgctgc tggtgctctc | 420 |
| cctcccattc aagatggtcc tgtcccaggt acagtccccc ttcccgtccc tgtgcaccct | 480 |
| ggtggagtgc ctttacttcg tcagcatgta cggaagcgtc ttcaccatct gcttcatcag | 540 |
| catggaccgg ttcttggcca tccgttaccc gctactggtg agccacctcc ggtcccccag | 600 |
| gaagatcttt gggatctgct gcaccatctg ggtcctggtg tggaccggaa gcatccctat | 660 |
| ctacagtttc catgggaaag tggaaaaata catgtgcttc cacaacatgt ctgatgatac | 720 |
| ctggagcgcc aaggtctccc tcccgctgga ggtgtttggc ttcctccttc ccatgggcat | 780 |
| catgggcttc tgctgctcca ggagcatcca catcctgctg ggccgccgag accacaccca | 840 |
| ggactgggtg cagcagaaag cctgcatcta cagcatcgca gccagcctgg ctgtcttcgt | 900 |
| ggtctccttc ctcccagccc acctggggtt cttcctgcag ttcctggtga gaaacagctt | 960 |
| tatcgtagag tgcagagcca agcagagcat cagcttcttc ttgcaattgt ccatgtgttt | 1020 |
| ctccaacgtc aactgctgcc tggatgtttt ctgctactac tttgtcatca aagaattccg | 1080 |
| catgaacatc agggcccacc ggccttccag ggtccagctg gtcctgcagg acaccacgat | 1140 |
| ctcccggggc taacggaagg acatcctgtt caggggaaga aagccctggc cctgaattct | 1200 |
| ggtaacggat atcgcgttcc agggttttga tgtggtggga tgatccgcac catcttcact | 1260 |
| gatgtgcttc cctttgatgc ccattgagtg ccagctttgc tcattatacc ccaaagacct | 1320 |
| tttttccact gcccagacag cttataccac ccagtgttca gggatctctg aagaacccac | 1380 |
| agaccaggtg aattactgat ttctaagtcc aaaaactata gagcagaaga attgagaaag | 1440 |
| agaatgagac catgtcaaca aggctgtttc caactccccc catettcctg ttcgactggg | 1500 |
| aggttctgga aagaaagaga gagagagaaa agaggtggat ggagggaagg gtgaggaaga | 1560 |
| gagcgtcaat gaaggggtgg aagtggatgt ggaggagtga ccagaccaga ccagaccaga | 1620 |
| ccagagggat cggaggctca ggccccctga gctgggcctc taagaaccac agccacagag | 1680 |
| tcaggctctg cagacccaga ggtaaggctg cctggctgcc ccaaacccca gacactcctc | 1740 |
| tccttgcctg caggccgccc tctcccatgg ccaccctaga cagagacacc acccgtgata | 1800 |
| ctctgaggga gccagacttt cacatgcaac ggggcatttg gggctggttc gggtcagtgt | 1860 |
| ctacgggtgg tggatttagt gaccaccctc ctcccacttc tttctctgca cgaggagttg | 1920 |
| ggagcctgcc tggtggagtt gagtgatgcc cgtcatgtgc acttgggtct accaatgtgc | 1980 |
| ttaaggccag atgggcttcc taagagacaa gggcgggcgg acctaaccaa ctatcatccc | 2040 |
| cagcgacgtt caaacataaa atcttatcta tgtgcagcat gactctctcc aggtgacaga | 2100 |
| aagggctcta gacagctgag aggacctgat catgtaggga gggacgggga ggggagccag | 2160 |
| gacccaggag ctgcatggct gtaagaggaa ggtccttgga gggtatcagc agtctcagtg | 2220 |
| tgatgtgaca actatggacc tgtgggcgcg ccctggggac ccctgaccca tcccaaaaca | 2280 |
| ctgtggaaag aagggcctct tctccgtggg gacccgtgtc tgtgggagcg ggcacaggtc | 2340 |
| catgctgcag gccactacgg ctgctcggca ggagggccga gagtttggac ccacatagct | 2400 |
| gcaacccaag ctggcccata accagcagtg gggcccagca gcacaggagg ggcggacggt | 2460 |
| tagctttgga aaaaatgact tgattaatct gattcgccaa ataaatagat tcatgtgatg | 2520 |
| ccttgtgaaa aaaaaaaaaa aaaaaaaaaa aaaaaaaaaa aaaaaaaaaa aaaaaaaaaa | 2580 |
| aaaaaaaaaa aaaaaaaaaa aaaaaaaaaa aaaaaaaaaa aaaaaaaaa | 2629 |

//

1. A method of predicting the metastatic potential or determining a metastasis prognosis of breast cancer cells in a biological sample from an individual, the method comprising assessing the level of GPR55 expression in the cells.

2. A method as claimed in claim 1 which comprises the steps of:
(a) contacting the sample with a binding agent that specifically binds to GPR55;
(b) detecting the amount of GPR55 that binds to the binding agent;
(c) optionally comparing the amount of GPR55 to a predetermined cut-off value, and thereby determining the metastatic potential of the cancer cells.

3. A method as claimed in claim 2 wherein the agent is an antibody.

4. A method as claimed in claim 1 which comprises the steps of:
(a) determining the amount of GPR55 mRNA in the sample;
(b) optionally comparing the amount of GPR55 mRNA in the test sample to a predetermined value; and thereby determining the metastatic potential of the cancer cells.

5. A method as claimed in claim 4 wherein the amount of GPR55 mRNA in the sample is determined by RT-qPCR.

6. A method as claimed in any one of claims 1 to 5 wherein the biological sample is one taken from a primary tumour.

7. A method as claimed in any one of claims 1 to 6 wherein the level of GPR55 expression is normalised compared to a reference gene or protein in the cells.

8. A method as claimed in any one of claims 1 to 7 wherein the level of GPR55 expression is compared to a control value.

9. A method as claimed in any one of claims 1 to 8 for use in diagnosing the risk or likelihood of metastases in the individual or for use in selecting an individual for therapy against metastases.

10. A method for monitoring the progression of breast cancer in an individual, comprising the steps of:
(a) assessing the level of GPR55 expression in a biological sample obtained from the individual at a first point;
(b) repeating step (a) using a biological sample obtained from the individual at a subsequent point in time; and
(c) comparing the level of GPR55 expression detected in step (b) to the amount detected in step (a) and thereby monitoring the progression of the cancer in the individual.

11. A method as claimed in claim 10 for monitoring the risk of metastases in the individual over time or for monitoring the efficacy of an anticancer therapy which the individual is undergoing.

12. A test kit for use in a method according to any one of claims 1 to 11, wherein the kit comprises:
(a) a nucleic acid molecule comprising at least 30 contiguous nucleotides of the GPR55 nucleotide sequence; and
(b) means for detecting binding of the nucleic acid molecule to the GPR55 mRNA in a sample;
or the kit comprises:
(a) an antibody which selectively binds GPR55;
(b) means for detecting binding of the antibody to GPR55.

13. A method of screening for an agent for use in a method of treatment of breast cancer in an individual, wherein the agent is a GPR55 inhibitor, the method comprising the step of assessing the binding of said substance to the GPR55 receptor or the step of assessing its ability to inhibit expression of the GPR55.

14. A method as claimed in claim 13 which comprises:
(i) exposing cells or membranes comprising GPR55 to a test compound for a predetermined length of time;
(ii) detecting the activity or expression of GPR55;
(iii) comparing the activity or expression of the GPR55 in the cells or membranes treated with the compound relative to activity or expression found in control cells or membranes that were not treated with the compound; and
(iv) selecting agents which decrease activity or decrease expression of GPR55 relative to the controls.

15. A process of producing a pharmaceutical composition for use in a method of treatment of breast cancer in an individual, wherein the process comprises:
(i) selecting the agent by use of the method of claim 13 or claim 14;
(ii) producing said agent;
(iii) formulating the agent into a pharmaceutical composition.

16. A pharmaceutical composition comprising a GPR55 inhibitor for use in a method of treatment of breast cancer in a individual.

17. A method of treatment of breast cancer in a patient, which method comprises the step of contacting the tumour with a GPR55 inhibitor.

18. Use of a GPR55 inhibitor in the preparation of a medicament for the treatment of breast cancer.

19. A process, composition, method or use as claimed in any one of claims 13 to 18 wherein the GPR55 inhibitor exerts its effect in one or more the following ways:
(a) decreasing the expression of GPR55;
(b) directly antagonising GPR55 or increasing receptor desensitisation or receptor breakdown;
(c) reducing interaction between GPR55 and its endogenous ligands;
(d) reducing GPR55 mediated intracellular signalling; and\or
(e) competing with endogenous GPR55 for endogenous ligand binding.

20. A process, composition, method or use as claimed in any one of claims 13 to 19 wherein the treatment is such as to inhibit metastasis of a primary breast cancer tumour.

21. A process, composition, method or use as claimed in any one of claims 13 to 20 wherein the treatment is such as to inhibit metastasis of a primary breast cancer tumour to bone.

22. A process, composition, method or use as claimed in any one of claims 13 to 21 wherein the treatment is adjuvant therapy such as to reduce the risk of cancer recurrence.

23. A process, composition, method or use as claimed in any one of claims 13 to 22 wherein the treatment is a combination treatment wherein the GPR55 inhibitor is used either simultaneously or sequentially in combination with a further therapeutic intervention for the treatment of breast cancer.

24. A process, composition, method or use as claimed in claim 23 wherein the further therapeutic intervention comprises administration of doxorubicin; cyclophosphamide; paclitaxel; and\or trastuzumab.

25. A process, composition, method or use as claimed in claim 23 wherein the further therapeutic intervention is a surgical intervention.


SEQUENCE LISTING (2 sequences)

SEQ ID NO: 1 - DNA, 2629 bp, Homo sapiens
caggcctggaggagaaatgacatctctcagccctctcagctgcaccggaccaccaacagttgtgattcaatgggcatgaattgctgtgtgatgctggggaggtgtttgtgattcttgacaaagtcatttgaatccatcacttcaagagagtgaaaggagccccgtctgatctgttggtgttgtaggaagaaacatgagtcagcaaaacaccagtggggactgcctgtttgacggtgtcaacgagctgatgaaaaccctacagtttgcagtccacatccccaccttcgtcctgggcctgctcctcaacctgctggccatccatggcttcagcaccttccttaagaacaggtggcccgattatgctgccacctccatctacatgatcaacctggcagtctttgacctgctgctggtgctctccctcccattcaagatggtcctgtcccaggtacagtcccccttcccgtccctgtgcaccctggtggagtgcctttacttcgtcagcatgtacggaagcgtcttcaccatctgcttcatcagcatggaccggttcttggccatccgttacccgctactggtgagccacctccggtcccccaggaagatctttgggatctgctgcaccatctgggtcctggtgtggaccggaagcatccctatctacagtttccatgggaaagtggaaaaatacatgtgcttccacaacatgtctgatgatacctggagcgccaaggtcttcttcccgctggaggtgtttggcttcctccttcccatgggcatcatgggcttctgctgctccaggagcatccacatcctgctgggccgccgagaccacacccaggactgggtgcagcagaaagcctgcatctacagcatcgcagccagcctggctgtcttcgtggtctccttcctcccagtccacctggggttcttcctgcagttcctggtgagaaacagctttatcgtagagtgcagagccaagcagagcatcagcttcttcttgcaattgtccatgtgtttctccaacgtcaactgctgcctggatgttttctgctactactttgtcatcaaagaattccgcatgaacatcagggcccaccggccttccagggtccagctggtcctgcaggacaccacgatctcccggggctaacggaaggacatcctgttcaggggaagaaagccctggccctgaattctggtaacggatatcgcgttccagggttttgatgtggtgggatgatccgcaccatcttcactgatgtgcttccctttgatgcccattgagtgccagctttgctcattataccccaaagaccttttttccactgcccagacagcttataccacccagtgttcagggatctctgaagaacccacagaccaggtgaattactgatttctaagtccaaaaactatagagcagaagaattgagaaagagaatgagaccatgtcaacaaggctgtttccaactctccccattttcctgttcgactgggaggttctggaaagaaagagagagagagaaaagaggtggatggagggaagggtgaggaagagagcgtcaatgaaggggtggaagtggatgtggaggagtgaccagaccagaccagaccagaccagagggatcggaggctcaggccccctgagctgggcctctaagaaccacagccacagagtcaggctctgcagacccagaggtaaggctgcctggctgccccaaaccccagacactcctctccttgcctgcaggctgccctctcccatggccaccctagacagagacaccacccgtgatactctgagggagccagactttcacatgcaacggggcatttggggctggttcgggtcagtgtctacgggtggtggatttagtgaccaccctcctcccacttctttctctgcacgaggagttgggagcctgcctggtggagttgagtgatgcccgtcatgtgcacttgggtctaccaatgtgcttaaggccagatgggcttcctaagagacaagggtgggtggacctaaccaactatcatccccagcgatgttcaaacataaaatcttatctatgtgcagcatgactctctccaggtgacagaaagggctctagacagctgagaggacctgatcatgtagggagggacggggaggggagccaggacccaggagctgcatggctgtaagaggaaggtccttggagggtatcagcagtctcagtgtgatgtgacaactatggacctgtgggtgtgccctggggacccctgacccatcccaaaacactgtggaaagaagggcctcttctccgtggggacccgtgtctgtgggagcgggcacaggtccatgctgcaggccactacggctgctcggcaggagggccgagagtttggacccacatagctgcaacccaagctggcccataatcagcagtggggcccagcagcacaggaggggcggacggttagctttggaaaaaatgacttgattaatctgattcgccaaataaatagattcatgtgatgccttgtgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - protein, 319 aa, Homo sapiens
MSQQNTSGDCLFDGVNELMKTLQFAVHIPTFVLGLLLNLLAIHGFSTFLKNRWPDYAATSIYMINLAVFDLLLVLSLPFKMVLSQVQSPFPSLCTLVECLYFVSMYGSVFTICFISMDRFLAIRYPLLVSHLRSPRKIFGICCTIWVLVWTGSIPIYSFHGKVEKYMCFHNMSDDTWSAKVFFPLEVFGFLLPMGIMGFCCSRSIHILLGRRDHTQDWVQQKACIYSIAASLAVFVVSFLPVHLGFFLQFLVRNSFIVECRAKQSISFFLQLSMCFSNVNCCLDVFCYYFVIKEFRMNIRAHRPSRVQLVLQDTTISRG